(19) United States
(12) Patent Application Publication
(10) Pub. No.: US 2024/0336605 A1
(43) Pub. Date: Oct. 10, 2024

(54) METHOD FOR PRODUCING SULFONE DERIVATIVE AS HERBICIDE

(71) Applicant: KUMIAI CHEMICAL INDUSTRY CO., LTD., Tokyo (JP)

(72) Inventor: Shinki Tani, Tokyo (JP)

(73) Assignee: KUMIAI CHEMICAL INDUSTRY CO., LTD., Tokyo (JP)

(21) Appl. No.: 18/270,799

(22) PCT Filed: Mar. 10, 2022
(86) PCT No.: PCT/JP2022/010671

§ 371 Date: Jul. 3, 2023

(30) Foreign Application Priority Data
Mar. 11, 2021 (JP) 2021-038948

(51) Int. Cl. C07D 413/12

(52) U.S. Cl. CPC C07D 413/12

(57) ABSTRACT

The present invention provides an industrially preferred method for producing a sulfone derivative that is useful as an herbicide. This process is for producing a compound of the formula (2). In the method, a compound of the formula (1) is reacted with an oxidizer in the presence of a metal catalyst and the presence of a carboxylic acid to produce the compound of the formula (2).

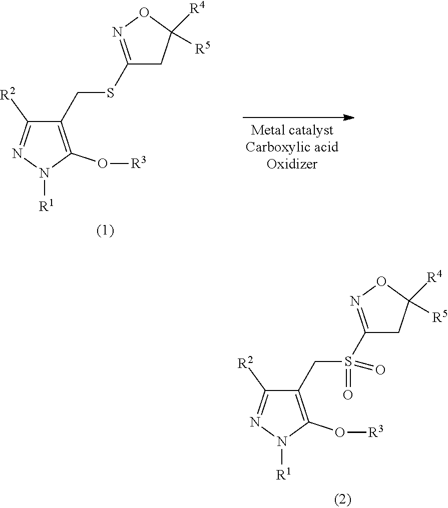

### TECHNICAL FIELD

The present invention relates to a process for producing a sulfone derivative useful as a herbicide, that is, a compound of the following formula (2):

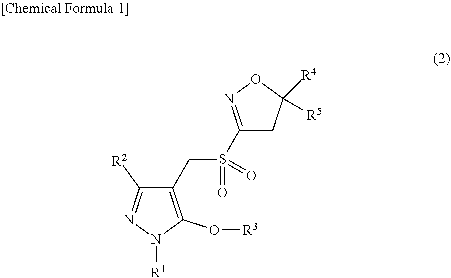

wherein R¹, R², R³, R⁴ and R⁵ are as described herein.

### BACKGROUND ART

It is known that sulfone derivatives of the above formula (2) have a herbicidal activity as disclosed in WO 2002/062770 A1 (Patent Document 1). Among them, pyroxasulfone is well known as a superior herbicide.

As a process for producing the compound of the formula (2), a process by the oxidation of a sulfide derivative, i.e., a compound of the following formula (1) has been known, which is shown below.

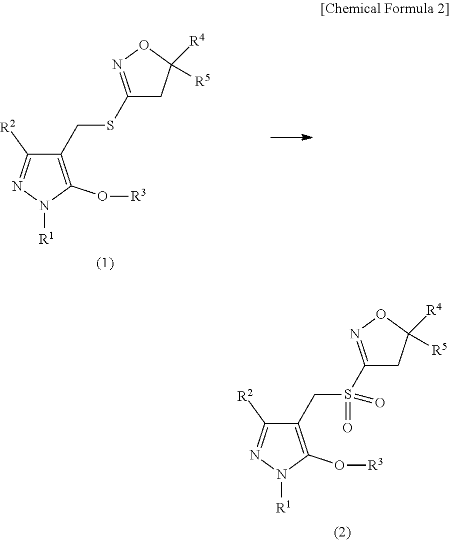

As shown in the following scheme, in Reference Example 3 in WO 2004/013106 A1 (Patent Document 2) is disclosed a process for producing 3-(5-difluoromethoxy-1-methyl-3-trifluoromethyl-1H-pyrazol-4-ylmethanesulfonyl)-5,5-dimethyl-2-isoxazoline (2-a) (pyroxasulfone) by oxidizing 3-(5-difluoromethoxy-1-methyl-3-trifluoromethyl-1H-pyrazol-4-ylmethylthio)-5,5-dimethyl-2-isoxazoline (1-a) (ISFP) with m-chloroperoxybenzoic acid (mCPBA).

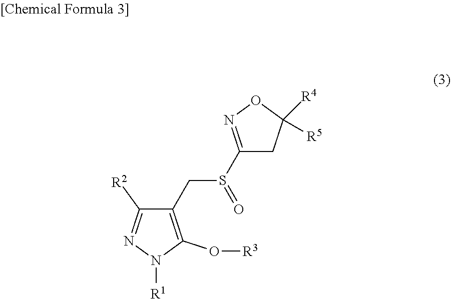

In the process for producing the compound of the formula (2) from the compound of the formula (1), m-chloroperoxybenzoic acid (mCPBA) disclosed in WO 2004/013106 A1 (Patent Document 2) is expensive for industrial use and has problems in handling and waste. Therefore, the process for producing described in WO 2004/013106 A1 (Patent Document 2) is not practical for production on an industrial scale.

In addition, in the process for producing the compound of the formula (2) (sulfone derivative: SO₂ derivative) from the compound of the formula (1) (sulfide derivative: S derivative), there is a possibility that the reaction stops at a sulfoxide derivative (SO derivative) that is an intermediate of the oxidation reaction, i.e., a compound of the following formula (3):

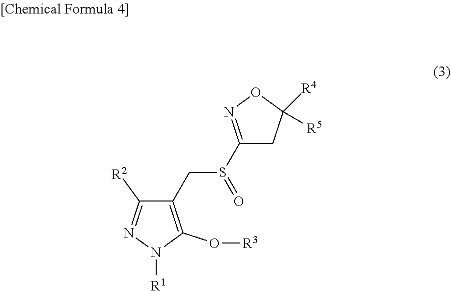

wherein R¹, R², R³, R⁴ and R⁵ are as described herein.

Therefore, the compound of the formula (3) sometimes remains in the product as a by-product. The compound of the formula (3) that has contaminated a product such as a herbicide leads to the possibility of reduced quality and crop injury. However, the physical and chemical properties of the compound of the formula (3) are very similar to those of the compound of the formula (2), so that it is difficult to separate the compound of the formula (3) to purify the compound of the formula (2). Therefore, regarding the process for producing the compound of the formula (2) from the compound of the formula (1), there has been desired a process in which the oxidation reaction sufficiently proceeds and substantially no compound of the formula (3) remains in the product.

In Example 9C in Patent Document 3 (JP 2013-512201 A), a process for producing pyroxasulfone using acetic acid is disclosed. However, the process disclosed in Example 9C in JP 2013-512201 A has a disadvantage that a large amount of the intermediate (sulfoxide derivative: SO derivative) of the formula (3) remains. See Reference Example 1 herein.

Patent Document 3 (JP 2013-512201 A) corresponds to Patent Document 4 (US 2012/264947 A1).

In Example 5 in CN 111574511 A (Patent Document 5), a process for producing pyroxasulfone using acetic acid is disclosed. However, the process disclosed in Example 5 in CN 111574511 A has been found to be non-reproducible and has the disadvantage of leaving a large amount of the intermediate (sulfoxide derivative: SO derivative) of the formula (3). See Reference Example 2 herein.

In WO 2021/002484 A2 (Patent Document 6), a process for producing pyroxasulfone is disclosed. This process is a superior process that solves the above problems. However, there is still room for improvement such that the process described in WO 2021/002484 A2 is generally performed at a relatively high temperature.

In addition, there is still room for improvement in the processes of the conventional arts because the reaction rate may be relatively slow.

### CITATION LIST

### Patent Document

Patent Document 1: WO 2002/062770 A1
Patent Document 2: WO 2004/013106 A1
Patent Document 3: JP 2013-512201 A
Patent Document 4: US 2012/264947 A1
Patent Document 5: CN 111574511 A
Patent Document 6: WO 2021/002484 A2

### SUMMARY OF THE INVENTION

### Problems to be Solved by the Invention

It is an object of the present invention to provide a process for producing the compound of the formula (2) from the compound of the formula (1) that affords a product containing the compound of the formula (3) in a sufficiently low percentage, is superior in yield, and industrially advantageous.

### Means for Solving the Problems

In view of the circumstances as described above, the present inventors have diligently studied processes for producing a compound of the formula (2). As a result, it has been unexpectedly found that the problem can be solved by providing the following processes for producing a compound of the formula (2). Based on this finding, the present inventors have completed the present invention. That is, in one embodiment, the present invention is as follows.

[A-1] A process for producing a compound of the formula (2), comprising:
a step of reacting a compound of the formula (1) with an oxidizing agent in the presence of a metal catalyst and the presence of a carboxylic acid;

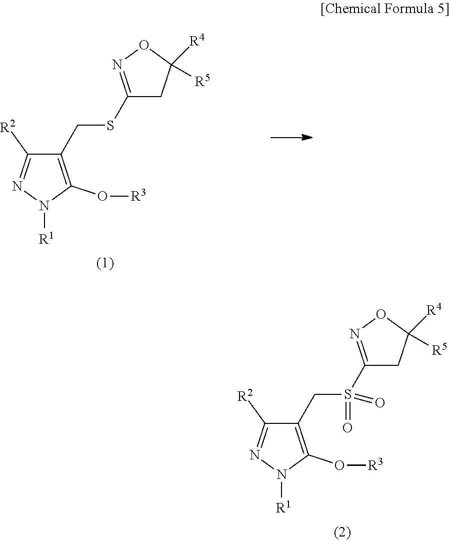

wherein in the formula (1) and (2),
R¹, R² and R³ are each independently a (C1-C6)alkyl optionally substituted with one or more substituents, a (C3-C6)cycloalkyl optionally substituted with one or more substituents, a (C2-C6)alkenyl optionally substituted with one or more substituents, a (C2-C6)alkynyl optionally substituted with one or more substituents, or a (C6-C10)aryl optionally substituted with one or more substituents, and
R⁴ and R⁵ are each independently a (C1-C6)alkyl optionally substituted with one or more substituents, a (C3-C6)cycloalkyl optionally substituted with one or more substituents, a (C2-C6)alkenyl optionally substituted with one or more substituents, a (C2-C6)alkynyl optionally substituted with one or more substituents, a (C1-C6)alkoxy optionally substituted with one or more substituents; or a (C6-C10)aryl optionally substituted with one or more substituents, or R⁴ and R⁵, together with the carbon atom to which they are attached, form a 3- to 12-membered carbocyclic ring, wherein the formed ring is optionally substituted with one or more substituents.

[A-2] The process according to [A-1], wherein the reaction is performed at 35° C. or higher.

[A-3] The process according to [A-1], wherein the reaction is performed at above 35° C.

[A-4] The process according to [A-1], wherein the reaction is performed at 40° C. or higher.

[A-5] The process according to [A-1], wherein the reaction is performed at 45° C. or higher.

[A-6] The process according to [A-1], wherein the reaction is performed at 50° C. or higher.

[A-7] The process according to any one of [A-1] to [A-6], wherein the reaction is performed at 60° C. or lower.

[A-8] The process according to any one of [A-1] to [A-6], wherein the reaction is performed at below 60° C.

[A-9] The process according to any one of [A-1] to [A-6], wherein the reaction is performed at 55° C. or lower.

[A-10] The process according to any one of [A-1] to [A-6], wherein the reaction is performed at below 55° C.

[A-11] The process according to any one of [A-1] to [A-6], wherein the reaction is performed at 50° C. or lower.

[A-12] The process according to any one of [A-1] to [A-6], wherein the reaction is performed at under 50° C.

[A-13] The process according to any one of [A-1] to [A-6], wherein the reaction is performed at 45° C. or lower.

[A-14] The process according to any one of [A-1] to [A-6], wherein the reaction is performed at 40° C. or lower.

[A-15] The process according to any one of [A-1] to [A-6], wherein the reaction is performed at 35° C. or lower.

[A-16] The process according to any one of [A-1] to [A-15], wherein an amount of the carboxylic acid is 0.05 mol or more (preferably 0.1 mol or more) based on 1 mol of the compound of the formula (1).

[A-17] The process according to any one of [A-1] to [A-15], wherein an amount of the carboxylic acid is 0.5 mol or more (1 mol or more, 2 mol or more or 3 mol or more) based on 1 mol of the compound of the formula (1).

[A-18] The process according to any one of [A-1] to [A-15], wherein an amount of the carboxylic acid is 5 mol or more based on 1 mol of the compound of the formula (1).

[A-19] The process according to any one of [A-1] to [A-15], wherein an amount of the carboxylic acid is 8 mol or more (or 9 mol or more) based on 1 mol of the compound of the formula (1).

[A-20] The process according to any one of [A-1] to [A-15], wherein an amount of the carboxylic acid is 10 mol or more (or 12 mol or more) based on 1 mol of the compound of the formula (1).

[A-21] The process according to any one of [A-1] to [A-15], wherein an amount of the carboxylic acid is 15 mol or more based on 1 mol of the compound of the formula (1).

[A-22] The process according to any one of [A-1] to [A-15], wherein an amount of the carboxylic acid is 18 mol or more (or 20 mol or more) based on 1 mol of the compound of the formula (1).

[A-23] The process according to any one of [A-1] to [A-15], wherein an amount of the carboxylic acid is 26 mol or more (preferably 28 mol or more and more preferably 30 mol or more) based on 1 mol of the compound of the formula (1).

[A-24] The process according to any one of [A-1] to [A-15], wherein an amount of the carboxylic acid is 32 mol or more based on 1 mol of the compound of the formula (1).

[A-25] The process according to any one of [A-1] to [A-15], wherein an amount of the carboxylic acid is 35 mol or more based on 1 mol of the compound of the formula (1).

[A-26] The process according to any one of [A-1] to [A-25], wherein an amount of the carboxylic acid is 90 mol or less (preferably 70 mol or less) based on 1 mol of the compound of the formula (1).

[A-27] The process according to any one of [A-1] to [A-25], wherein an amount of the carboxylic acid is 55 mol or less based on 1 mol of the compound of the formula (1).

[A-28] The process according to any one of [A-1] to [A-25], wherein an amount of the carboxylic acid is 10 mol or less (or 9 mol or less) based on 1 mol of the compound of the formula (1).

[A-29] The process according to any one of [A-1] to [A-25], wherein an amount of the carboxylic acid is 5 mol or less based on 1 mol of the compound of the formula (1).

[A-30] The process according to any one of [A-1] to [A-29], wherein an amount of the carboxylic acid is 0.3 liters or more (preferably 0.5 liters or more) based on 1 mol of the compound of the formula (1).

[A-31] The process according to any one of [A-1] to [A-29], wherein an amount of the carboxylic acid is 0.8 liters or more (preferably 1.0 liter or more) based on 1 mol of the compound of the formula (1).

[A-32] The process according to any one of [A-1] to [A-29], wherein an amount of the carboxylic acid is 1.2 liters or more (preferably 1.5 liters or more) based on 1 mol of the compound of the formula (1).

[A-33] The process according to any one of [A-1] to [A-29], wherein an amount of the carboxylic acid is 1.8 liters or more (preferably 2.0 liters or more) based on 1 mol of the compound of the formula (1).

[A-34] The process according to any one of [A-1] to [A-33], wherein an amount of the carboxylic acid is 5 liters or less (preferably 3 liters or less) based on 1 mol of the compound of the formula (1).

[A-35] The process according to any one of [A-1] to [A-33], wherein an amount of the carboxylic acid is 2.0 liters or less (preferably 1.0 liter or less) based on 1 mol of the compound of the formula (1).

[A-36] The process according to any one of [A-1] to [A-33], wherein an amount of the carboxylic acid is 0.9 liters or less (preferably 0.8 liters or less) based on 1 mol of the compound of the formula (1).

[A-37] The process according to any one of [A-1] to [A-33], wherein an amount of the carboxylic acid is 0.5 liters or less (0.3 liters or less or 0.2 liters or less) based on 1 mol of the compound of the formula (1).

[A-38] The process according to any one of [A-1] to [A-37], wherein the reaction is performed in the absence of an organic solvent.

[A-39] The process according to any one of [A-1] to [A-37], wherein the reaction is performed in the presence or absence of an organic solvent.

[A-40] The process according to any one of [A-1] to [A-37], wherein the reaction is performed in the presence of an organic solvent.

[A-41] The process according to [A-39] or [A-40], wherein the organic solvent is an organic solvent having an acceptor number of 5 to 45.

[A-42] The process according to [A-39] or [A-40], wherein the organic solvent is an organic solvent having an acceptor number of 7 to 42.

[A-43] The process according to [A-39] or [A-40], wherein the organic solvent is an organic solvent having a relative permittivity of 1 to 45.

[A-44] The process according to [A-39] or [A-40], wherein the organic solvent is an organic solvent having a relative permittivity of 4 to 40.

[A-45] The process according to [A-39] or [A-40], wherein the organic solvent is an organic solvent having a Rohrschneider polarity parameter of 1 to 7 (preferably 3 to 6).

[A-46] The process according to [A-39] or [A-40], wherein the organic solvent is an organic solvent excluding carboxylic acids.

[A-47] The process according to [A-39] or [A-40], wherein the organic solvent is an organic solvent excluding a carboxylic acid of the formula (a);

A-COOH (a) Chemical Formula 6

wherein A is as described herein.

[A-48] The process according to [A-39] or [A-40], wherein the organic solvent is selected from the group consisting of aromatic hydrocarbon derivatives, halogenated aliphatic hydrocarbons, alcohols, nitriles, carboxylic acid esters, ethers, ketones, amides, ureas and sulfones.

[A-49] The process according to [A-39] or [A-40], wherein the organic solvent is selected from the group consisting of aromatic hydrocarbon derivatives, halogenated aliphatic hydrocarbons, alcohols, nitriles, carboxylic acid esters and amides.

[A-50] The process according to [A-39] or [A-40], wherein the organic solvent is selected from the group consisting of benzene optionally substituted with 1 to 3 (preferably 1 or 2) substituents selected from (C1-C4)alkyl and chlorine atom, and (C1-C4)alkane optionally substituted with 1 to 10 halogen atoms (preferably chlorine atoms), (C1-C6)alcohol, (C2-C5)alkane nitrile, (C1-C4)alkyl (C2-C6)carboxylates and N,N-di((C1-C4)alkyl) (C1-C4)alkane amide.

[A-51] The process according to [A-39] or [A-40], wherein the organic solvent is selected from the group consisting of toluene, xylene, chlorobenzene, dichlorobenzene, dichloromethane, 1,2-dichloroethane, methanol, ethanol, propanol, 2-propanol, butanol, sec-butanol, isobutanol, tert-butanol, pentanol, sec-amyl alcohol, 3-pentanol, 2-methyl-1-butanol, isoamyl alcohol, tert-amyl alcohol, hexanol and isomers thereof, cyclohexanol, acetonitrile, methyl acetate, ethyl acetate, propyl acetate, isopropyl acetate, butyl acetate and isomers thereof, pentyl acetate and isomers thereof, hexyl acetate and isomers thereof, N,N-dimethylformamide (DMF), N,N-dimethylacetamide (DMAC), and N,N-diethylacetamide.

[A-52] The process according to [A-39] or [A-40], wherein the organic solvent is selected from the group consisting of toluene, xylene, chlorobenzene, dichlorobenzene, dichloromethane, methanol, ethanol, propanol, 2-propanol, butanol, sec-butanol, isobutanol, tert-butanol, sec-amyl alcohol, acetonitrile, methyl acetate, ethyl acetate, propyl acetate, isopropyl acetate, butyl acetate and isomers thereof, N,N-dimethylformamide (DMF), N,N-dimethylacetamide (DMAC) and N,N-diethylacetamide.

[A-53] The process according to [A-39] or [A-40], wherein the organic solvent is selected from the group consisting of halogenated aliphatic hydrocarbons, alcohols, nitriles, carboxylic acid esters and amides.

[A-54] The process according to [A-39] or [A-40], wherein the organic solvent is selected from the group consisting of (C1-C4)alkane optionally substituted with 1 to 10 halogen atoms (preferably chlorine atoms), (C1-C6)alcohol, (C2-C5)alkane nitrile, (C1-C4)alkyl (C2-C6)carboxylate and N,N-di((C1-C4)alkyl) (C1-C4)alkane amide.

[A-55] The process according to [A-39] or [A-40], wherein the organic solvent is selected from the group consisting of dichloromethane, 1,2-dichloroethane, methanol, ethanol, propanol, 2-propanol, butanol, sec-butanol, isobutanol, tert-butanol, pentanol, sec-amyl alcohol, 3-pentanol, 2-methyl-1-butanol, isoamyl alcohol, tert-amyl alcohol, hexanol and isomers thereof, cyclohexanol, acetonitrile, methyl acetate, ethyl acetate, propyl acetate, isopropyl acetate, butyl acetate and isomers thereof, N,N-dimethylformamide (DMF), N,N-dimethylacetamide (DMAC) and N,N-diethylacetamide.

[A-56] The process according to [A-39] or [A-40], wherein the organic solvent is selected from the group consisting of dichloromethane, methanol, ethanol, propanol, 2-propanol, butanol, sec-butanol, isobutanol, tert-butanol, sec-amyl alcohol, acetonitrile, methyl acetate, ethyl acetate, propyl acetate, isopropyl acetate, butyl acetate and isomers thereof, N,N-dimethylformamide (DMF), N,N-dimethylacetamide (DMAC) and N,N-diethylacetamide.

[A-57] The process according to [A-39] or [A-40], wherein the organic solvent is selected from the group consisting of halogenated aliphatic hydrocarbons, alcohols and nitriles.

[A-58] The process according to [A-39] or [A-40], wherein the organic solvent is selected from the group consisting of (C1-C4)alkane optionally substituted with 1 to 10 halogen atoms (preferably chlorine atoms), (C1-C6)alcohol and (C2-C5)alkane nitrile.

[A-59] The process according to [A-39] or [A-40], wherein the organic solvent is selected from the group consisting of dichloromethane, 1,2-dichloroethane, chloroform, methanol, ethanol, propanol, 2-propanol, butanol, sec-butanol, isobutanol, tert-butanol, pentanol, sec-amyl alcohol, 3-pentanol, 2-methyl-1-butanol, isoamyl alcohol, tert-amyl alcohol, hexanol and isomers thereof, cyclohexanol, and acetonitrile.

[A-60] The process according to [A-39] or [A-40], wherein the organic solvent is selected from the group consisting of dichloromethane, methanol, ethanol, propanol, 2-propanol, butanol, sec-butanol, isobutanol, tert-butanol, sec-amyl alcohol and acetonitrile.

[A-61] The process according to [A-39] or [A-40], wherein the organic solvent is selected from the group consisting of dichloromethane, methanol and acetonitrile.

[A-62] The process according to [A-39] or [A-40], wherein the organic solvent is dichloromethane.

[A-63] The process according to any one of [A-39] and [A-40], wherein the organic solvent is (C1-C6)alcohol.

[A-64] The process according to [A-39] or [A-40], wherein the organic solvent is selected from the group consisting of methanol, ethanol, propanol, 2-propanol, butanol, sec-butanol, isobutanol, tert-butanol and tert-amyl alcohol.

[A-65] The process according to [A-39] or [A-40], wherein the organic solvent is methanol.

[A-66] The process according to [A-39] or [A-40], wherein the organic solvent is acetonitrile.

[A-67] The process according to any one of [A-1] to [A-66], wherein the reaction is performed in the presence of a solvent and the solvent comprises a carboxylic acid.

[A-68] The process according to any one of [A-1] to [A-66], wherein the reaction is performed in the presence of a solvent and a carboxylic acid is used as the solvent.

[A-69] The process according to any one of [A-1] to [A-66], wherein the reaction is performed in the presence of a solvent and the solvent is a mixed solvent of a carboxylic acid and an organic solvent excluding carboxylic acids.

[A-70] The process according to any one of [A-1] to [A-66], wherein the reaction is performed in the presence of a solvent and a mixed solvent of a carboxylic acid and an organic solvent excluding carboxylic acids is used as the solvent.

[A-71] The process according to any one of [A-1] to [A-70], wherein an amount of the organic solvent is 0.1 liters or more (preferably 0.2 liters or more) based on 1 mol of the compound of the formula (1).

[A-72] The process according to any one of [A-1] to [A-70], wherein an amount of the organic solvent is 0.3 liters or more based on 1 mol of the compound of the formula (1).

[A-73] The process according to any one of [A-1] to [A-70], wherein an amount of the organic solvent is 0.5 liters or more based on 1 mol of the compound of the formula (1).

[A-74] The process according to any one of [A-1] to [A-70], wherein an amount of the organic solvent is 0.8 liters or more based on 1 mol of the compound of the formula (1).

[A-75] The process according to any one of [A-1] to [A-74], wherein an amount of the organic solvent is 3 liters or less based on 1 mol of the compound of the formula (1).

[A-76] The process according to any one of [A-1] to [A-74], wherein an amount of the organic solvent is 2 liters or less based on 1 mol of the compound of the formula (1).

[A-77] The process according to any one of [A-1] to [A-74], wherein an amount of the organic solvent is 1 liter or less based on 1 mol of the compound of the formula (1).

[A-78] The process according to any one of [A-1] to [A-37], wherein the reaction is performed in the presence of a solvent and the solvent is a carboxylic acid.

[A-79] The process according to any one of [A-1] to [A-78], wherein the reaction is performed in the presence of a solvent and the solvent comprises water.

[A-80] The process according to any one of [A-1] to [A-78], wherein the reaction is performed in the presence of a water solvent.

[A-81] The process according to [A-79] or [A-80], wherein an amount of the water solvent is more than 0 (zero) liter based on 1 mol of the compound of the formula (1).

[A-82] The process according to [A-79] or [A-80], wherein an amount of the water solvent is 0.1 liters or more based on 1 mol of the compound of the formula (1).

[A-83] The process according to [A-79] or [A-80], wherein an amount of the water solvent is 0.18 liters or more based on 1 mol of the compound of the formula (1).

[A-84] The process according to [A-79] or [A-80], wherein an amount of the water solvent is 0.5 liters or less based on 1 mol of the compound of the formula (1).

[A-85] The process according to [A-79] or [A-80], wherein an amount of the water solvent is 0.3 liters or less based on 1 mol of the compound of the formula (1).

[A-86] The process according to [A-79] or [A-80], wherein an amount of the water solvent is 0.25 liters or less based on 1 mol of the compound of the formula (1).

[A-87] The process according to any one of [A-1] to [A-86], wherein the carboxylic acid is a carboxylic acid of the formula (a):

A-COOH (a) Chemical Formula 7

wherein A is hydrogen, a (C1-C6)alkyl optionally substituted with one or more substituents, a (C3-C6)cycloalkyl optionally substituted with one or more substituents, a (C2-C6)alkenyl optionally substituted with one or more substituents, or a (C2-C6)alkynyl optionally substituted with one or more substituents.

[A-88] The process according to [A-87], wherein A is a (C1-C4)alkyl optionally substituted with one or more substituents.

[A-89] The process according to [A-87], wherein A is a (C1-C4)alkyl optionally substituted with 1 to 9 halogen atoms.

[A-90] The process according to [A-87], wherein A is a (C1-C4)alkyl optionally substituted with 1 to 9 fluorine or chlorine atoms.

[A-91] The process according to [A-87], wherein A is a (C1-C4)alkyl optionally substituted with 1 to 9 fluorine atoms.

[A-92] The process according to [A-87], wherein A is a (C1-C4)alkyl optionally substituted with 1 to 9 chlorine atoms.

[A-93] The process according to [A-87], wherein A is a (C1-C4)alkyl.

[A-94] The process according to any one of [A-1] to [A-86], wherein the carboxylic acid is selected from the group consisting of acetic acid, difluoroacetic acid, trifluoroacetic acid, dichloroacetic acid and trichloroacetic acid.

[A-95] The process according to any one of [A-1] to [A-86], wherein the carboxylic acid is selected from the group consisting of difluoroacetic acid, trifluoroacetic acid, dichloroacetic acid and trichloroacetic acid.

[A-96] The process according to any one of [A-1] to [A-86], wherein the carboxylic acid is selected from the group consisting of acetic acid, dichloroacetic acid and trichloroacetic acid.

[A-97] The process according to any one of [A-1] to [A-86], wherein the carboxylic acid is acetic acid.

[A-98] The process according to any one of [A-1] to [A-86], wherein the carboxylic acid is selected from the group consisting of dichloroacetic acid and trichloroacetic acid.

[A-99] The process according to any one of [A-1] to [A-86], wherein the carboxylic acid is dichloroacetic acid.

[A-100] The process according to any one of [A-1] to [A-86], wherein the carboxylic acid is trichloroacetic acid.

[A-101] The process according to any one of [A-1] to [A-100], wherein a metal of the metal catalyst is a transition metal.

[A-102] The process according to any one of [A-1] to [A-100], wherein a metal of the metal catalyst is selected from the group consisting of Groups 5 and 6 of the periodic table.

[A-103] The process according to any one of [A-1] to [A-100], wherein the metal catalyst is selected from the group consisting of a tungsten catalyst and a molybdenum catalyst.

[A-104] The process according to any one of [A-1] to [A-100], wherein the metal catalyst is a tungsten catalyst.

[A-105] The process according to any one of [A-1] to [A-100], wherein the metal catalyst is a molybdenum catalyst.

[A-106] The process according to any one of [A-1] to [A-100], wherein the metal catalyst is selected from the group consisting of tungstic acid, tungstic acid salts, molybdic acid and molybdic acid salts.

[A-107] The process according to any one of [A-1] to [A-100], wherein the metal catalyst is selected from the group consisting of tungstic acid, a tungstic acid alkali metal salt, a tungstic acid ammonium salt, molybdic acid, a molybdic acid alkali metal salt and an ammonium molybdate salt.

[A-108] The process according to any one of [A-1] to [A-100], wherein the metal catalyst is selected from the group consisting of sodium tungstate and ammonium molybdate.

[A-109] The process according to any one of [A-1] to [A-100], wherein the metal catalyst is a tungstic acid alkali metal salt (preferably sodium tungstate).

[A-110] The process according to any one of [A-1] to [A-100], wherein the metal catalyst is ammonium molybdate.

[A-111] The process according to any one of [A-1] to [A-100], wherein the metal catalyst is selected from the group consisting of sodium tungstate dihydrate and an ammonium molybdate tetrahydrate salt.

[A-112] The process according to any one of [A-1] to [A-100], wherein the metal catalyst is sodium tungstate dihydrate.

[A-113] The process according to any one of [A-1] to [A-100], wherein the metal catalyst is an ammonium molybdate tetrahydrate salt.

[A-114] The process according to any one of [A-1] to [A-113], wherein the oxidizing agent is hydrogen peroxide.

[A-115] The process according to any one of [A-1] to [A-113], wherein the hydrogen peroxide is a 10 to 70 wt % aqueous hydrogen peroxide solution.

[A-116] The process according to any one of [A-1] to [A-113], wherein the hydrogen peroxide is a 20 to 65 wt % aqueous hydrogen peroxide solution.

[A-117] The process according to any one of [A-1] to [A-113], wherein the hydrogen peroxide is a 25 to 65 wt % aqueous hydrogen peroxide solution.

[A-118] The process according to any one of [A-1] to [A-117], wherein the reaction is performed in the presence or absence of an acid catalyst.

[A-119] The process according to any one of [A-1] to [A-118], wherein the reaction is performed in the presence of an acid catalyst and the acid catalyst is sulfuric acid.

[A-120] The process according to any one of [A-1] to [A-119], wherein
R¹ is a (C1-C4)alkyl,
R² is a (C1-C4)perfluoroalkyl,
R³ is a (C1-C4)alkyl optionally substituted with 1 to 9 fluorine atoms, and
R⁴ and R⁵ are each independently a (C1-C4)alkyl.

[A-121] The process according to any one of [A-1] to [A-119], wherein
R¹ is methyl,
R² is trifluoromethyl,
R³ is difluoromethyl, and
R⁴ and R⁵ are methyl.

In another embodiment, the present invention is as follows.

[B-1] A process for producing a compound of the formula (2), comprising:
a step of reacting a compound of the formula (1) with an oxidizing agent in the presence of a metal catalyst and the presence of a carboxylic acid,
wherein the reaction is performed at above 35° C.;

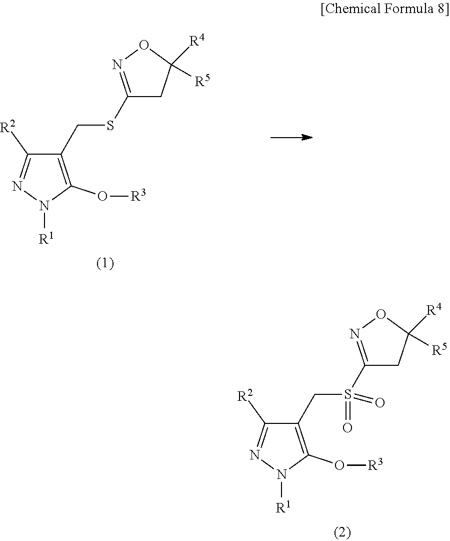

wherein in the formula (1) and (2),
R¹, R² and R³ are each independently a (C1-C6)alkyl optionally substituted with one or more substituents, a (C3-C6)cycloalkyl optionally substituted with one or more substituents, a (C2-C6)alkenyl optionally substituted with one or more substituents, a (C2-C6)alkynyl optionally substituted with one or more substituents, or a (C6-C10)aryl optionally substituted with one or more substituents, and
R⁴ and R⁵ are each independently a (C1-C6)alkyl optionally substituted with one or more substituents, a (C3-C6)cycloalkyl optionally substituted with one or more substituents, a (C2-C6)alkenyl optionally substituted with one or more substituents, a (C2-C6)alkynyl optionally substituted with one or more substituents, a (C1-C6)alkoxy optionally substituted with one or more substituents; or a (C6-C10)aryl optionally substituted with one or more substituents, or R⁴ and R⁵, together with the carbon atom to which they are attached, form a 3- to 12-membered carbocyclic ring, wherein the formed ring is optionally substituted with one or more substituents.

[B-2] The process according to [B-1], wherein the reaction is performed at 40° C. or higher.

[B-3] The process according to [B-1], wherein the reaction is performed at 45° C. or higher.

[B-4] The process according to any one of [B-1] to [B-3], wherein the reaction is performed at 60° C. or lower.

[B-5] The process according to any one of [B-1] to [B-3], wherein the reaction is performed at 55° C. or lower.

[B-6] A process for producing a compound of the formula (2), comprising:
a step of reacting a compound of the formula (1) with an oxidizing agent in the presence of a metal catalyst and the presence of a carboxylic acid,
wherein an amount of the carboxylic acid is 18 mol or more based on 1 mol of the compound of the formula (1);

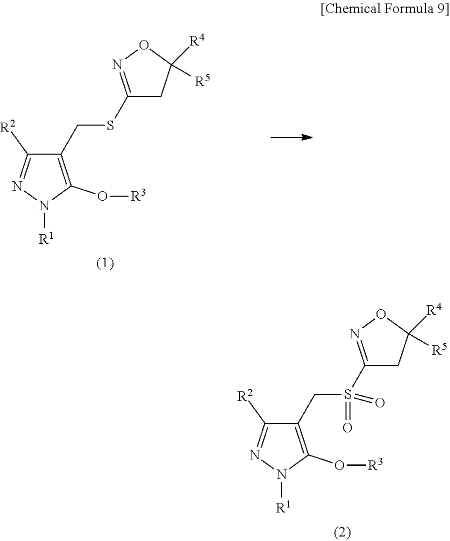

wherein in the formula (1) and (2),
R¹, R² and R³ are each independently a (C1-C6)alkyl optionally substituted with one or more substituents, a (C3-C6)cycloalkyl optionally substituted with one or more substituents, a (C2-C6)alkenyl optionally substituted with one or more substituents, a (C2-C6)alkynyl optionally substituted with one or more substituents, or a (C6-C10)aryl optionally substituted with one or more substituents, and
R⁴ and R⁵ are each independently a (C1-C6)alkyl optionally substituted with one or more substituents, a (C3-C6)cycloalkyl optionally substituted with one or more substituents, a (C2-C6)alkenyl optionally substituted with one or more substituents, a (C2-C6)alkynyl optionally substituted with one or more substituents, a (C1-C6)alkoxy optionally substituted with one or more substituents; or a (C6-C10)aryl optionally substituted with one or more substituents, or R⁴ and R⁵, together with the carbon atom to which they are attached, form a 3- to 12-membered carbocyclic ring, wherein the formed ring is optionally substituted with one or more substituents.

[B-7] The process according to [B-6], wherein the amount of the carboxylic acid is 30 mol or more based on 1 mol of the compound of the formula (1).

[B-8] The process according to [B-6], wherein the amount of the carboxylic acid is 35 mol or more based on 1 mol of the compound of the formula (1).

[B-9] A process for producing a compound of the formula (2), comprising:
a step of reacting a compound of the formula (1) with an oxidizing agent in the presence of a metal catalyst and the presence of a carboxylic acid, wherein the reaction is performed in the presence of an organic solvent excluding carboxylic acids,

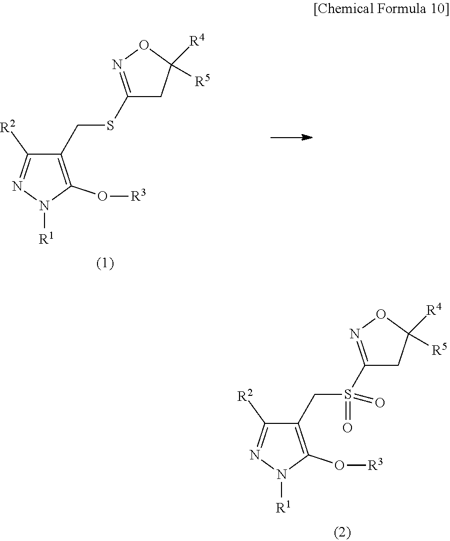

wherein in the formula (1) and (2),
R¹, R² and R³ are each independently a (C1-C6)alkyl optionally substituted with one or more substituents, a (C3-C6)cycloalkyl optionally substituted with one or more substituents, a (C2-C6)alkenyl optionally substituted with one or more substituents, a (C2-C6)alkynyl optionally substituted with one or more substituents, or a (C6-C10)aryl optionally substituted with one or more substituents, and
R⁴ and R⁵ are each independently a (C1-C6)alkyl optionally substituted with one or more substituents, a (C3-C6)cycloalkyl optionally substituted with one or more substituents, a (C2-C6)alkenyl optionally substituted with one or more substituents, a (C2-C6)alkynyl optionally substituted with one or more substituents, a (C1-C6)alkoxy optionally substituted with one or more substituents; or a (C6-C10)aryl optionally substituted with one or more substituents, or R⁴ and R⁵, together with the carbon atom to which they are attached, form a 3- to 12-membered carbocyclic ring, wherein the formed ring is optionally substituted with one or more substituents.

[B-10] The process according to [B-9], wherein the organic solvent is selected from the group consisting of aromatic hydrocarbon derivatives, halogenated aliphatic hydrocarbons, alcohols, nitriles, carboxylic acid esters and amides.

[B-11] The process according to [B-9], wherein the organic solvent is selected from the group consisting of halogenated aliphatic hydrocarbons, alcohols and nitriles.

[B-12] The process according to [B-9], wherein the organic solvent is selected from the group consisting of (C1-C4)alkane optionally substituted with 1 to 10 halogen atoms, (C1-C6)alcohol and (C2-C5)alkane nitrile.

[B-13] The process according to [B-9], wherein the organic solvent is selected from the group consisting of dichloromethane, methanol and acetonitrile.

[B-14] The process according to any one of [B-1] to [B-13], wherein the carboxylic acid is a carboxylic acid of the formula (a):

A-COOH (a) Chemical Formula 11

wherein A is hydrogen, a (C1-C6)alkyl optionally substituted with one or more substituents, a (C3-C6)cycloalkyl optionally substituted with one or more substituents, a (C2-C6)alkenyl optionally substituted with one or more substituents, or a (C2-C6)alkynyl optionally substituted with one or more substituents.

[B-15] The process according to any one of [B-1] to [B-13], wherein the carboxylic acid is acetic acid.

[B-16] The process according to any one of [B-1] to [B-13], wherein the carboxylic acid is dichloroacetic acid.

[B-17] The process according to any one of [B-1] to [B-13], wherein the carboxylic acid is trichloroacetic acid.

[B-18] The process according to any one of [B-1] to [B-17], wherein the metal catalyst is selected from the group consisting of a tungsten catalyst and a molybdenum catalyst.

[B-19] The process according to any one of [B-1] to [B-17], wherein the metal catalyst is a tungsten catalyst.

[B-20] The process according to any one of [B-1] to [B-17], wherein the metal catalyst is a molybdenum catalyst.

[B-21] The process according to any one of [B-1] to [B-20], wherein the oxidizing agent is hydrogen peroxide.

[B-22] The process according to any one of [B-1] to [B-21], wherein
R¹ is a (C1-C4)alkyl,
R² is a (C1-C4)perfluoroalkyl,
R³ is a (C1-C4)alkyl optionally substituted with 1 to 9 fluorine atoms, and
R⁴ and R⁵ are each independently a (C1-C4)alkyl.

[B-23] The process according to any one of [B-1] to [B-21], wherein
R¹ is methyl,
R² is trifluoromethyl,
R³ is difluoromethyl, and
R⁴ and R⁵ are methyl.

In still another embodiment, the present invention is as follows.

[C-1] A process for producing a compound of the formula (2), comprising:
a step of reacting a compound of the formula (1) with an oxidizing agent in the presence of a metal catalyst and the presence of a carboxylic acid to produce the compound of the formula (2),

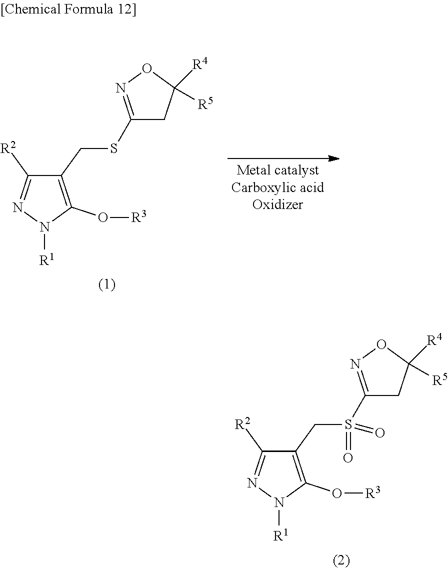

wherein in the formula (1) and (2)
R¹, R² and R³ are each independently a (C1-C6)alkyl optionally substituted with one or more substituents, a (C3-C6)cycloalkyl optionally substituted with one or more substituents, a (C2-C6)alkenyl optionally substituted with one or more substituents, a (C2-C6)alkynyl optionally substituted with one or more substituents, or a (C6-C10)aryl optionally substituted with one or more substituents, and
R⁴ and R⁵ are each independently a (C1-C6)alkyl optionally substituted with one or more substituents, a (C3-C6)cycloalkyl optionally substituted with one or more substituents, a (C2-C6)alkenyl optionally substituted with one or more substituents, a (C2-C6)alkynyl optionally substituted with one or more substituents, a (C1-C6)alkoxy optionally substituted with one or more substituents; or a (C6-C10)aryl optionally substituted with one or more substituents, or R⁴ and R⁵, together with the carbon atom to which they are attached, form a 3- to 12-membered carbocyclic ring, wherein the formed ring is optionally substituted with one or more substituents.

[C-2] The process according to [C-1], wherein the reaction is performed at above 35° C.

[C-3] The process according to [C-1], wherein the reaction is performed at 40° C. or higher.

[C-4] The process according to [C-1], wherein the reaction is performed at 45° C. or higher.

[C-5] The process according to any one of [C-1] to [C-4], wherein an amount of the carboxylic acid used is more than 26 mol based on 1 mol of the compound of the formula (1).

[C-6] The process according to any one of [C-1] to [C-4], wherein an amount of the carboxylic acid used is 30 mol or more based on 1 mol of the compound of the formula (1).

[C-7] The process according to any one of [C-1] to [C-4], wherein an amount of the carboxylic acid used is 35 mol or more based on 1 mol of the compound of the formula (1).

[C-8] The process according to any one of [C-1] to [C-7], wherein the carboxylic acid is a carboxylic acid of the formula (a):

A-COOH (a) Chemical Formula 13

wherein A is hydrogen, an optionally substituted (C1-C6)alkyl, a (C3-C6)cycloalkyl optionally substituted with one or more substituents, a (C2-C6)alkenyl optionally substituted with one or more substituents, or a (C2-C6)alkynyl optionally substituted with one or more substituents.

[C-9] The process according to [C-8], wherein A is an optionally substituted (C1-C4)alkyl.

[C-10] The process according to any one of [C-1] to [C-7], wherein the carboxylic acid is acetic acid.

[C-11] The process according to any one of [C-1] to [C-10], wherein the metal catalyst is a tungsten catalyst or a molybdenum catalyst.

[C-12] The process according to any one of [C-1] to [C-10], wherein the metal catalyst is a tungsten catalyst.

[C-13] The process according to any one of [C-1] to [C-10], wherein the metal catalyst is a molybdenum catalyst.

[C-14] The process according to any one of [C-1] to [C-13], wherein the oxidizing agent is hydrogen peroxide.

[C-15] The process according to any one of [C-1] to [C-14], wherein
R¹ is a (C1-C4)alkyl,
R² is a (C1-C4)perfluoroalkyl,
R³ is a (C1-C4)alkyl optionally substituted with 1 to 9 fluorine atoms, and
R⁴ and R⁵ are each independently a (C1-C4)alkyl.

[C-16] The process according to any one of [C-1] to [C-14], wherein
R¹ is methyl,
R² is trifluoromethyl,
R³ is difluoromethyl, and
R⁴ and R⁵ are methyl.

### Effects of the Invention

The present invention provides a process for producing a compound of the formula (2) (sulfone derivative: SO₂ derivative) from a compound of the formula (1) (sulfide derivative: S derivative), in which the ratio of a compound of the formula (3) (sulfoxide derivative: SO derivative) in a product is sufficiently low and the process is industrially preferable.

The compound of the formula (2) produced by the process of the present invention contains substantially no compound of the formula (3) which may cause a reduction in quality as a herbicide and crop injury, and therefore it is useful as the herbicide.

### DESCRIPTION OF EMBODIMENTS

Hereinafter, the present invention will be described in detail.

The symbols and terms described in the present description will be explained.

Herein, the following abbreviations and prefixes may be used, and their meanings are as follows:
Me: methyl
Et: ethyl
Pr, n-Pr and Pr-n: propyl (i.e., normal propyl)
i-Pr and Pr-i: isopropyl
Bu, n-Bu and Bu-n: butyl (i.e., normal butyl)
s-Bu and Bu-s: sec-butyl (i.e., secondary butyl)
i-Bu and Bu-i: isobutyl
t-Bu and Bu-t: tert-butyl (i.e., tertiary butyl)
Ph: phenyl
n-: normal
s- and sec-: secondary
i- and iso-: iso
t- and tert-: tertiary
c- and cyc-: cyclo
o-: ortho
m-: meta
p-: para

The term “nitro” means the substituent “—NO₂”.

The term “cyano” or “nitrile” means the substituent “—CN”.

The term “hydroxy” means the substituent “—OH”.

The term “amino” means the substituent “—NH₂”.

(Ca-Cb) means that the number of carbon atoms is a to b. For example, “(C1-C4)” in “(C1-C4)alkyl” means that the number of carbon atoms in the alkyl is 1 to 4.

Herein, it is to be interpreted that generic terms such as “alkyl” include both a straight chain and a branched chain such as butyl and tert-butyl. Meanwhile, for example, the specific term “butyl” refers to straight “normal butyl” and does not refer to branched “tert-butyl”. Branched chain isomers such as “tert-butyl” are referred to specifically when intended.

Examples of the halogen atom include fluorine atom, chlorine atom, bromine atom and iodine atom.

The (C1-C6)alkyl means a straight or branched alkyl having 1 to 6 carbon atoms. Examples of the (C1-C6)alkyl include, but are not limited to, methyl, ethyl, propyl, isopropyl, butyl, sec-butyl, isobutyl, tert-butyl, pentyl and hexyl.

The (C1-C4)alkyl means a straight or branched alkyl having 1 to 4 carbon atoms. Examples of the (C1-C4)alkyl include appropriate examples of the examples of the (C1-C6)alkyl above-mentioned.

The (C3-C6)cycloalkyl means a cycloalkyl having 3 to 6 carbon atoms. Examples of the (C3-C6)cycloalkyl are cyclopropyl, cyclobutyl, cyclopentyl and cyclohexyl.

The (C2-C6)alkenyl means a straight or branched alkenyl having 2 to 6 carbon atoms. Examples of the (C2-C6)alkenyl include, but are not limited to, vinyl, 1-propenyl, isopropenyl, 2-propenyl, 1-butenyl, 1-methyl-1-propenyl, 2-methyl-1-propenyl, 2-butenyl, 3-butenyl, 1,3-butadienyl, 1-pentenyl and 1-hexenyl.

The (C2-C6)alkynyl means a straight or branched alkynyl having 2 to 6 carbon atoms. Examples of the (C2-C6)alkynyl include, but are not limited to, ethynyl, 1-propynyl, 2-propynyl, 1-butynyl, 1-methyl-2-propynyl, 2-butynyl, 3-butynyl, 1-pentynyl and 1-hexynyl.

Examples of the (C6-C10)aryl are phenyl, 1-naphthyl and 2-naphthyl.

The (C1-C6)haloalkyl means a straight or branched alkyl having 1 to 6 carbon atoms which is substituted with 1 to 13 halogen atoms which are the same or different from each other (here, the halogen atoms have the same meaning as defined above). Examples of the (C1-C6)haloalkyl include, but are not limited to, fluoromethyl, chloromethyl, bromomethyl, difluoromethyl, dichloromethyl, trifluoromethyl, trichloromethyl, chlorodifluoromethyl, bromodifluoromethyl, 2-fluoroethyl, 1-chloroethyl, 2-chloroethyl, 2,2,2-trifluoroethyl, pentafluoroethyl, 3-fluoropropyl, 3-chloropropyl, 2-chloro-1-methylethyl, 2,2,3,3,3-pentafluoropropyl, 2,2,2-trifluoro-1-trifluoromethylethyl, heptafluoropropyl, 1,2,2,2-tetrafluoro-1-trifluoromethylethyl, 4-fluorobutyl, 4-chlorobutyl, 2,2,3,3,4,4,4-heptafluorobutyl, nonafluorobutyl, 1,1,2,3,3,3-hexafluoro-2-trifluoromethylpropyl, 2,2,2-trifluoro-1,1-di(trifluoromethyl)ethyl, undecafluoropentyl and tridecafluorohexyl.

The (C1-C4)perfluoroalkyl means a straight or branched alkyl having 1 to 4 carbon atoms, wherein all hydrogen atoms are substituted with fluorine atoms. Examples of the (C1-C4)perfluoroalkyl are trifluoromethyl (i.e., —CF₃), pentafluoroethyl (i.e., —CF₂CF₃), heptafluoropropyl (i.e., —CF₂CF₂CF₃), 1,2,2,2-tetrafluoro-1-trifluoromethylethyl (i.e., —CF(CF₃)₂), nonafluorobutyl, (i.e., —CF₂CF₂CF₂CF₃), 1,2,2,3,3,3-hexafluoro-1-trifluoromethylpropyl (i.e., —CF(CF₃) CF₂CF₃), 1,1,2,3,3,3-hexafluoro-2-trifluoromethylpropyl (i.e., —CF₂CF(CF₃)₂) and 2,2,2-trifluoro-1,1-di(trifluoromethyl) ethyl (i.e., —C(CF₃)₃).

Examples of the (C1-C4)alkyl optionally substituted with 1 to 9 fluorine atoms include, but are not limited to, fluoromethyl (i.e., —CH₂F), difluoromethyl (i.e., —CHF₂), trifluoromethyl (i.e., —CF₃), 2-fluoroethyl, 2,2,2-trifluoroethyl, pentafluoroethyl, 3-fluoropropyl, 2,2,3,3,3-pentafluoropropyl, 2,2,2-trifluoro-1-trifluoromethylethyl, heptafluoropropyl, 1,2,2,2-tetrafluoro-1-trifluoromethylethyl, 4-fluorobutyl, 2,2,3,3,4,4,4-heptafluorobutyl, nonafluorobutyl, 1,1,2,3,3,3-hexafluoro-2-trifluoromethylpropyl and 2,2,2-trifluoro-1,1-di(trifluoromethyl)ethyl.

The (C1-C6)alkoxy means a (C1-C6)alkyl-O—, wherein the (C1-C6)alkyl moiety has the same meaning as defined above. Examples of the (C1-C6)alkoxy include, but are not limited to, methoxy, ethoxy, propoxy, isopropoxy, butoxy, sec-butoxy, isobutoxy, tert-butoxy, pentyloxy, isopentyloxy, neopentyloxy and hexyloxy.

The cyclic hydrocarbon group means a cyclic group which is monocyclic or multicyclic, wherein all of the ring-constituting atoms are carbon atoms. In one embodiment, examples of the cyclic hydrocarbon group include, but are not limited to, a 3- to 14-membered (preferably 5- to 14-membered, more preferably 5- to 10-membered) cyclic hydrocarbon group which is aromatic or non-aromatic and is monocyclic, bicyclic or tricyclic. In another embodiment, examples of the cyclic hydrocarbon group include, but are not limited to, a 4- to 8-membered (preferably 5- to 6-membered) cyclic hydrocarbon group which is aromatic or non-aromatic and is monocyclic or bicyclic (preferably monocyclic). Examples of the cyclic hydrocarbon group include, but are not limited to, cycloalkyls and aryls. Examples of the cycloalkyl include the examples of the (C3-C6)cycloalkyl described above. The aryls are aromatic cyclic groups among the cyclic hydrocarbon groups as defined above. Examples of the aryl include the examples of the (C6-C10)aryl described above. The cyclic hydrocarbon group as defined or exemplified above may include a non-condensed cyclic group (e.g., a monocyclic group or a spirocyclic group) and a condensed cyclic group, when possible. The cyclic hydrocarbon group as defined or exemplified above may be either unsaturated, partially saturated or saturated, when possible. The cyclic hydrocarbon group as defined or exemplified above is also referred to as a carbocyclic ring group. The carbocyclic ring is a ring which corresponds to the cyclic hydrocarbon group as defined or exemplified above. Examples of the carbocyclic ring include, but are not limited to, cyclopropane, cyclobutane, cyclopentane, cyclohexane, cyclopentene and cyclohexene. Examples of the 3- to 12-membered carbocyclic ring are as described above.

Herein, there are no particular limitations on the “substituent(s)” for the phrase “optionally substituted with one or more substituents” as long as they are chemically acceptable and exhibit the effects of the present invention.

Herein, examples of the “substituent(s)” for the phrase “optionally substituted with one or more substituent(s)” include, but are not limited to, one or more substituents (preferably 1 to 4 substituents) selected independently from Substituent Group (I).

Substituent Group (I) is a group consisting of a halogen atom, a nitro group, a cyano group, a hydroxy group, an amino group, (C1-C6)alkyl, (C1-C6)haloalkyl, (C3-C6)cycloalkyl, (C2-C6)alkenyl, (C2-C6)alkynyl, (C1-C6)alkoxy, phenyl, and phenoxy, preferably a group consisting of a halogen atom, a nitro group, a cyano group, a hydroxy group, an amino group, and (C1-C4)alkyl, and more preferably a group consisting of a halogen atom, a hydroxy group, and (C1-C4)alkyl. Substituent Group (I) is still more preferably a group consisting of a halogen atom, and (C1-C4) alkyl.

Herein, the phrase “as described herein” and similar phrases incorporate by reference all applicable definitions and, if any, all of applicable examples, preferred examples, more preferred examples, still more preferred examples, and particularly preferred examples herein.

Herein, a compound having isomers includes all of isomers and any mixture thereof in any ratio. For example, xylene includes o-xylene, m-xylene, p-xylene and any mixture thereof in any ratio. For example, dichlorobenzene includes o-dichlorobenzene, m-dichlorobenzene, p-dichlorobenzene and any mixture thereof in any ratio.

Herein, the phrases “amount of . . . used” and “amount of . . . ” have the same meaning and can be replaced with each other as long as they exhibit the effects of the present invention.

Herein, the phrases “excluding . . . ” and “other than . . . ” can be replaced with each other.

Herein, the non-limiting term “comprise(s)/comprising” can each optionally be replaced by the limiting phrase “consist(s) of/consisting of”.

Unless otherwise stated, all technical and scientific terms used herein have the same meaning as commonly understood by a person skilled in the art to which the present disclosure belongs.

Unless otherwise indicated, it is understood that numbers used herein to express characteristics such as quantities, sizes, concentrations, and reaction conditions are modified by the term “about”. In some embodiments, disclosed numerical values are interpreted applying the reported number of significant digits and conventional rounding techniques. In some embodiments, disclosed numerical values are interpreted as containing certain errors necessarily resulting from the standard deviation found in their respective testing measurements.

### (Raw Material: Compound of Formula (1))

A compound of the formula (1) is used as a raw material. The compound of the formula (1) may be a known compound or may be produced from a known compound according to a known process. Particularly preferred specific examples of the compound of the formula (1) are as follows:

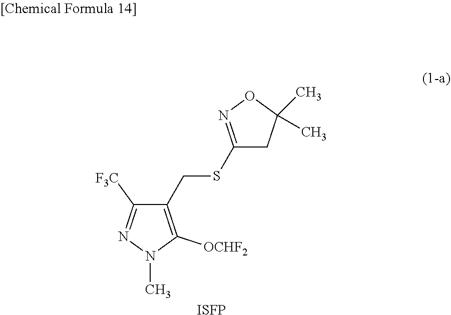

### (Product: Compound of Formula (2))

The product is a compound of the formula (2) corresponding to the compound of the formula (1) used as a raw material. Particularly preferred specific examples of the compound of the formula (2) are as follows:

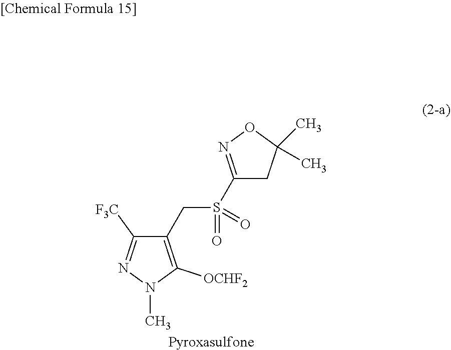

An intermediate of an oxidation reaction is a compound of the formula (3) corresponding to the compound of the formula (1) used as a raw material. Specific examples of the compound of the formula (3) are as follows:

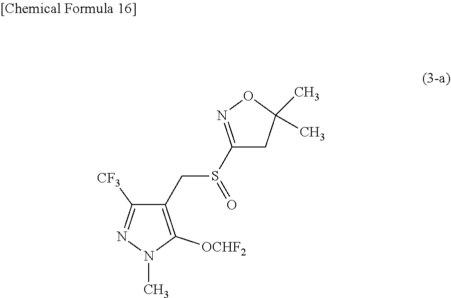 [SMILES: Cn1nc(C(F)(F)F)c(CS(=O)C2=NOC(C)(C)C2)c1OC(F)F]

As described above, in the process of producing the compound of the formula (2) (SO₂ derivative) from the compound of the formula (1) (S derivative), it is desired that the oxidation reaction sufficiently proceeds and the proportion of the compound of the formula (3) (SO derivative) in the product is sufficiently low. For example, in the reaction mixture after the reaction, the ratio of the compound of the formula (3) (SO derivative) is preferably 10% or less, more preferably 5% or less, still more preferably 4% or less, further preferably 3% or less, further preferably 2% or less, and further preferably 1% or less.

After the formula (1) is oxidized to obtain the formula (3), oxidation to the formula (2) may be performed.

### (Oxidizing Agent: Hydrogen Peroxide)

Examples of an oxidizing agent include, but are not limited to, peroxides, hypochlorites (e.g., sodium hypochlorite and potassium hypochlorite), manganates and manganese dioxide. Examples of the peroxide include, but are not limited to, hydrogen peroxide, peracid and salts thereof (e.g., peracetic acid), persulfuric acid and salts thereof (e.g., potassium peroxymonosulfate (Oxone (registered trademark)) and sodium peroxodisulfate). From the viewpoint of safety, economic efficiency, etc., preferable examples of the oxidizing agent include hydrogen peroxide.

The form of the hydrogen peroxide may be any form as long as the reaction proceeds. The form of the hydrogen peroxide can be appropriately selected by a person skilled in the art. However, in view of safety, danger and economic efficiency, etc., preferred examples of the form of the hydrogen peroxide include a 10 to 70 wt % aqueous hydrogen peroxide solution, more preferably a 20 to 65 wt % aqueous hydrogen peroxide solution, still more preferably a 25 to 65 wt % aqueous hydrogen peroxide solution, further preferably a 30 to 65 wt % aqueous hydrogen peroxide solution, and particularly preferably a 30 to 60 wt % aqueous hydrogen peroxide solution. Specific examples of the form of the hydrogen peroxide include, but are not limited to, a 30 wt % aqueous hydrogen peroxide solution, a 35 wt % aqueous hydrogen peroxide solution, a 50 wt % aqueous hydrogen peroxide solution and a 60 wt % aqueous hydrogen peroxide solution. The ranges of the concentrations of the hydrogen peroxide also include any combination of lower and upper limits of the ranges described herein.

The amount of the oxidizing agent (preferably hydrogen peroxide) used may be any amount as long as the reaction proceeds. The amount of the hydrogen peroxide used may be appropriately adjusted by a person skilled in the art. However, from the viewpoint of yield, suppression of by-products, economic efficiency, safety, risk, etc., the amount of the hydrogen peroxide used is, for example, 2 mol or more, preferably 2 to 8 mol, more preferably 2 to 6 mol, still more preferably 2 to 5 mol, and further preferably 2 to 4 mol, based on 1 mol of the compound of the formula (1) (raw material).

### (Metal Catalyst)

The metal catalyst may be any metal catalyst as long as the reaction proceeds. Examples of the metal catalyst include, but are not limited to, the following:
tungsten catalysts (e.g., tungstic acid, tungstic acid salts (e.g., sodium tungstates (including sodium tungstate dihydrate and sodium tungstate decahydrate), potassium tungstate, calcium tungstate, and ammonium tungstate), metal tungsten, tungsten oxides (e.g., tungsten(VI) oxide; tungsten(VI) oxide is also called tungsten trioxide), tungsten carbide, tungsten chlorides (e.g., tungsten(VI) chloride; tungsten(VI) chloride is also called tungsten hexachloride), tungsten bromides (e.g., tungsten(V) bromide), tungsten sulfides (e.g., tungsten(IV) sulfide; tungsten(IV) sulfide is also called tungsten disulfide), phosphotungstic acid and salts thereof (e.g., phosphotungstic acid, sodium phosphotungstate, and ammonium phosphotungstate), silicotungstic acid and salts thereof (e.g., silicotungstic acid and sodium silicotungstate), and a mixture thereof),
molybdenum catalysts (e.g., molybdic acid, molybdic acid salts, (e.g., sodium molybdate (including sodium molybdate dihydrate), potassium molybdate, ammonium molybdate (including ammonium molybdate tetrahydrate), metal molybdenum, molybdenum oxides (e.g., molybdenum(VI) oxide; molybdenum(VI) oxide is also called molybdenum trioxide), molybdate chlorides (molybdenum(V) chloride; molybdenum(V) chloride is also called molybdenum pentachloride), molybdenum sulfides (e.g., molybdenum(IV) sulfide; molybdenum(IV) sulfide is also called molybdenum disulfide), phosphomolybdic acid and salts thereof (e.g., phosphomolybdic acid, sodium phosphomolybdate, and ammonium phosphomolybdate), silicomolybdic acid and salts thereof (e.g., silicomolybdic acid and sodium silicomolybdate), bis(2,4-pentandionato)molybdenum(VI) dioxide, and a mixture thereof),
iron catalysts (e.g., iron(I) acetylacetoneate, iron(I) chloride and iron(I) nitrate, and a mixture thereof), manganese catalysts (e.g., potassium permanganate, manganese(II) oxide and manganese(II) chloride, and a mixture thereof),
vanadium catalysts (e.g., vanadyl acetylacetonate, vanadium(V) oxide, vanadium(V) oxytrichloride, vanadium(V) oxytriethoxyde and vanadium(V) oxytriisopropoxide, and a mixture thereof),
niobium catalysts (e.g., niobium carbide, niobium(V) chloride and niobium(V) pentaethoxyde, and a mixture thereof),
tantalum catalysts (e.g., tantalum carbide (TaC), tantalum(V) chloride (TaCl₅) and tantalum(V) pentaethoxyde (Ta(OEt)₅), and a mixture thereof),
titanium catalysts (e.g., titanium tetrachloride, titanium trichloride and titanium(IV) tetraisopropoxide, and a mixture thereof),
zirconium catalysts (e.g., zirconium dioxide, zirconium(I) chloride, zirconium(IV) chloride, zirconium chloride oxide, and a mixture thereof),
copper catalysts (e.g., copper(I) acetate, copper(II) acetate, copper(I) bromide and copper(I) iodide, and a mixture thereof),
thallium catalysts (e.g., thallium(I) nitrate, thallium(I) acetate and thallium (I) trifluoroacetate, and a mixture thereof).

Herein, an acid that can be in the form of a hydrate and a salt thereof may be in the form of a hydrate thereof, and any form is within the scope of the present invention.

Thus, for example, “sodium tungstate” encompasses “sodium tungstate dihydrate” and “sodium tungstate decahydrate”.

Herein, an acid that can be in the form of a polyacid and a salt thereof (e.g., tungstic acid and salts thereof) may be in the form of a polyacid, and any form is within the scope of the present invention.

The metal of the metal catalyst is preferably a transition metal. Specific examples thereof include Group 3 elements (Sc, Y, etc.), Group 4 elements (Ti, Zr, Hf), Group 5 elements (V, Nb, Ta), Group 6 elements (Cr, Mo, W), Group 7 elements (Mn, Tc, Re), Group 8 elements (Fe, Ru, Os), Group 9 elements (Co, Rh, Ir), Group 10 elements (Ni, Pd, Pt) and Group 11 elements (Cu, Ag, Au).

The transition metal of the metal catalyst is preferably a metal of Group 4, Group 5 or Group 6 on the periodic table, more preferably a metal of Group 5 or Group 6, and still more preferably a metal of Group 5.

Preferred examples of the metal catalyst are a tungsten catalyst and a molybdenum catalyst.

In one embodiment, a preferred example of the metal catalyst is a tungsten catalyst.

In another embodiment, a preferred example of the metal catalyst is a molybdenum catalyst.

In one embodiment, from the viewpoint of yield, suppression of by-products, economic efficiency, etc., preferred examples of the tungsten catalyst include the following:
tungstic acid, tungstic acid salts, metal tungsten, tungsten oxide, tungsten carbide, tungsten chloride, tungsten sulfide, phosphotungstic acid, silicotungstic acid and salts thereof, and a mixture thereof,
more preferably tungstic acid, tungstic acid salts, metal tungsten, tungsten oxide, tungsten carbide, tungsten chloride and salts thereof, and a mixture thereof,
still more preferably tungstic acid, tungstic acid salts, metal tungsten, tungsten oxide, tungsten carbide, and a mixture thereof,
further preferably tungstic acid, sodium tungstate, potassium tungstate, calcium tungstate, ammonium tungstate, metal tungsten, tungsten(VI) oxide, tungsten carbide, and a mixture thereof,
further preferably tungstic acid, sodium tungstate, metal tungsten, tungsten carbide, and a mixture thereof,
further preferably tungstic acid and sodium tungstate, and particularly preferably sodium tungstate.

From the viewpoint of yield, suppression of by-products, economic efficiency, etc., preferred examples of the molybdenum catalyst include the following:
molybdic acid, molybdic acid salts, metal molybdenum, molybdenum oxide, molybdenum carbide, molybdenum chloride, molybdenum sulfide, molybdenum bromide, phosphomolybdic acid, silicomolybdic acid and salts thereof, and a mixture thereof,
more preferably molybdic acid, molybdic acid salts, metal molybdenum, molybdenum carbide, molybdenum oxide, molybdenum chloride, and a mixture thereof,
still more preferably molybdic acid, sodium molybdate, potassium molybdate, ammonium molybdate, molybdenum(VI) oxide, molybdenum carbide, molybdenum(V) chloride, molybdenum(IV) sulfide, phosphomolybdic acid, sodium phosphomolybdate, ammonium phosphomolybdate, silicomolybdic acid, sodium silicomolybdate, and a mixture thereof,
further preferably molybdic acid, sodium molybdate, potassium molybdate, ammonium molybdate, molybdenum(VI) oxide, molybdenum(V) chloride, and a mixture thereof,
further preferably sodium molybdate, potassium molybdate and ammonium molybdate, and
particularly preferably ammonium molybdate.

From the viewpoint of yield, suppression of by-products, economic efficiency, etc., more preferred examples of the metal catalyst include the following: tungstic acid, sodium tungstate, potassium tungstate, calcium tungstate, ammonium tungstate, metal tungsten, tungsten oxide, tungsten carbide,
sodium molybdate, potassium molybdate and ammonium molybdate.

Still more preferred examples of the metal catalyst include the following:
tungstic acid, sodium tungstate,
sodium molybdate, potassium molybdate and ammonium molybdate.

Further preferred examples of the metal catalyst include the following:
sodium tungstate and ammonium molybdate.

In another embodiment, preferred metal catalysts are as described in [A-101] to [A-113] herein.

The metal catalyst may be used singly or in a combination of two or more kinds thereof in any ratio. The form of the metal catalyst may be any form as long as the reaction proceeds. The form thereof can be appropriately selected by a person skilled in the art. The amount of the metal catalyst used may be any amount as long as the reaction proceeds. The amount of the metal catalyst used may be appropriately adjusted by a person skilled in the art. However, from the viewpoint of yield, suppression of by-products, economic efficiency, etc., the use amount thereof is, for example, 0.001 to 0.1 mol, preferably 0.01 to 0.1 mol, more preferably 0.01 to 0.05 mol, and still more preferably 0.03 to 0.05 mol, based on 1 mol of the compound of the formula (1) (raw material).

In one embodiment, examples of a carboxylic acid include, but are not limited to, the following: Carboxylic acid of the formula (a);

A-COOH (a) Chemical Formula 17

wherein A is hydrogen, a (C1-C6)alkyl optionally substituted with one or more substituents, a (C3-C6)cycloalkyl optionally substituted with one or more substituents, a (C2-C6)alkenyl optionally substituted with one or more substituents, or a (C2-C6)alkynyl optionally substituted with one or more substituents.

From the viewpoint of yield, suppression of by-products, economic efficiency, etc., in one embodiment, preferred examples of A include a (C1-C4)alkyl optionally substituted with one or more substituents, more preferably a (C1-C4)alkyl optionally substituted with 1 to 9 halogen atoms, still more preferably a (C1-C4)alkyl optionally substituted with 1 to 9 substituents selected from fluorine and chlorine atoms, (in other words, a (C1-C4)alkyl optionally substituted with 1 to 9 fluorine or chlorine atoms.), and further preferably a (C1-C4)alkyl optionally substituted with chlorine atoms.

From the same viewpoint, in another embodiment, specific examples of preferred A include methyl, ethyl, monofluoromethyl, difluoromethyl, trifluoromethyl, monochloromethyl, dichloromethyl and trichloromethyl. More preferred specific examples of A include methyl, ethyl, trifluoromethyl and trichloromethyl. Still more preferred examples of A include methyl, trifluoromethyl and trichloromethyl. Further preferred examples of A include methyl, trifluoromethyl and trichloromethyl. From the same viewpoint, in still another embodiment, specific examples of preferred A include methyl, ethyl, difluoromethyl, trifluoromethyl, dichloromethyl and trichloromethyl. More preferred specific examples of A include methyl, difluoromethyl, trifluoromethyl, dichloromethyl and trichloromethyl. Still more preferred specific examples of A include methyl, dichloromethyl and trichloromethyl. In yet another embodiment, A is trifluoromethyl. In further embodiment, A is trichloromethyl. In still further embodiment, A is dichloromethyl. In yet further embodiment, A is methyl.

In yet further embodiment, examples of the carboxylic acid include, but are not limited to, the following: optionally substituted saturated or unsaturated aliphatic monocarboxylic acids (e.g., formic acid, acetic acid, propionic acid, butyric acid, monofluoroacetic acid, difluoroacetic acid, trifluoroacetic acid, monochloroacetic acid, dichloroacetic acid, trichloroacetic acid and lactic acid), optionally substituted saturated or unsaturated aliphatic dicarboxylic acids (e.g., oxalic acid, malonic acid, succinic acid, glutaric acid, adipic acid, fumaric acid, maleic acid, malic acid and tartaric acid), and optionally substituted saturated or unsaturated aliphatic tricarboxylic acids (e.g., citric acid). Herein, the formic acid is understood as one of the aliphatic monocarboxylic acids. Preferred specific examples of the carboxylic acid include, but are not limited to, the following: acetic acid, trifluoroacetic acid and trichloroacetic acid, and more preferably acetic acid. In yet further embodiment, preferred specific examples of the carboxylic acid include acetic acid, difluoroacetic acid, trifluoroacetic acid, dichloroacetic acid and trichloroacetic acid. More preferred specific examples of the carboxylic acid include acetic acid, dichloroacetic acid and trichloroacetic acid. Still more preferred specific examples of the carboxylic acid include acetic acid and dichloroacetic acid.

The amount of the carboxylic acid used is not particularly limited as long as the effects of the present invention are exhibited. However, from the viewpoint of yield, suppression of by-products, economic efficiency, etc., in one embodiment, the lower limit of the amount of the carboxylic acid used is, for example, more than 0 (zero) mol, preferably 0.01 mol or more, more preferably 0.05 mol or more, and still more preferably 0.1 mol or more, 0.3 mol or more, 0.5 mol or more, 1 mol or more, 2 mol or more, 3 mol or more or 5 mol or more, based on 1 mol of the compound of the formula (1) (raw material). In another embodiment, the lower limit of the amount of the carboxylic acid used is, for example, preferably 8 mol or more, 10 mol or more, 12 mol or more, 15 mol or more, 18 mol or more or 20 mol or more based on 1 mol of the compound of the formula (1) (raw material). In still another embodiment, the lower limit of the amount of the carboxylic acid used is, for example, 26 mol or more, preferably more than 26 mol, more preferably 27 mol or more or 28 mol or more, still more preferably 30 mol or more or 32 mol or more, and further preferably 35 mol or more, based on 1 mol of the compound of the formula (1) (raw material). From the same viewpoint as the above, in one embodiment, the upper limit of the amount of the carboxylic acid used is, for example, 90 mol or less, 70 mol or less or 55 mol or less based on 1 mol of the compound of the formula (1) (raw material). In another embodiment, the upper limit of the amount of the carboxylic acid used is, for example, 30 mol or less, 20 mol or less, 10 mol or less or 9 mol or less based on 1 mol of the compound of the formula (1) (raw material). In still another embodiment, the upper limit of the amount of the carboxylic acid used is, for example, 5 mol or less or 0.3 mol or less based on 1 mol of the compound of the formula (1) (raw material). The range of the amount of the carboxylic acid used is, for example, any appropriate combination of the lower and upper limits described above. For example, the combination of the lower and upper limits is as follows, but is not limited to: from the same viewpoint as the above, in one embodiment, the amount of the carboxylic acid used is, for example, more than 0 (zero) mol and 70 mol or less, more than 0 (zero) mol and 55 mol or less, or more than 0 (zero) mol and 30 mol or less, preferably 0.01 mol or more and 70 mol or less, 0.01 mol or more and 55 mol or less or 0.01 mol or more and 30 mol or less, more preferably 0.05 mol or more and 70 mol or less, 0.05 mol or more and 55 mol or less, or 0.05 mol or more and 30 mol or less, and still more preferably 0.1 mol or more and 70 mol or less, 0.1 mol or more and 55 mol or less, or 0.1 mol or more and 30 mol or less, based on 1 mol of the compound of the formula (1) (raw material). In another embodiment, the amount of the carboxylic acid used is, for example, more than 26 mol and 70 mol or less, or more than 26 mol and 55 mol or less, preferably 30 mol or more and 70 mol or less, or 30 mol or more and 55 mol or less, more preferably 35 mol or more and 70 mol or less, or 35 mol or more and 55 mol or less, based on 1 mol of the compound of the formula (1) (raw material). Depending on the purpose and the context, the carboxylic acids of the above amounts may be used as a solvent.

As long as the effects of the present invention are exhibited, some or all of the carboxylic acids may be salts and/or acid anhydrides.

### (Acid Catalyst)

The oxidation reaction of the present invention may be performed in the presence of an acid catalyst or in the absence of the acid catalyst. Whether or not to use the acid catalyst can be appropriately determined by a person skilled in the art. The acid of the acid catalyst is an acid excluding carboxylic acids. Examples of the acid catalyst include, but are not limited to, the following: mineral acids such as hydrochloric acid, sulfuric acid and nitric acid, sulfonic acids such as methanesulfonic acid, trifluoromethanesulfonic acid, benzenesulfonic acid and p-toluenesulfonic acid, phosphoric acids such as phosphoric acid, methyl phosphate, ethyl phosphate and phenyl phosphate, preferably sulfuric acid, phosphoric acid and phenyl phosphate, more preferably sulfuric acid and phenyl phosphate, and still more preferably sulfuric acid. The acid catalyst may be a salt thereof.

The acid catalyst may be used singly or in a combination of two or more kinds thereof in any ratio. The form of the acid catalyst may be any form as long as the reaction proceeds. Examples of the sulfuric acid include, but are not limited to, 50% to 98% sulfuric acid and 50% to 100% sulfuric acid, and preferably 90% to 98% sulfuric acid and 90% to 100% sulfuric acid (concentrated sulfuric acid). The form of the acid catalyst can be appropriately selected by a person skilled in the art. The amount of the acid catalyst used may be any amount as long as the reaction proceeds. The amount of the acid catalyst used may be appropriately adjusted by a person skilled in the art. However, from the viewpoint of yield, suppression of by-products, economic efficiency, etc., in one embodiment, the amount of the acid catalyst used is, for example, 0 (zero) to 0.5 mol, more than 0 (zero) and 0.5 mol or less, 0.005 to 0.5 mol, 0.01 to 0.5 mol, or 0.05 to 0.5 mol, and preferably 0 (zero) to 0.2 mol, more than 0 (zero) and 0.2 mol or less, 0.005 to 0.2 mol, 0.01 to 0.2 mol, or 0.05 to 0.2 mol, based on 1 mol of the compound of the formula (1) (raw material).

### (Phase Transfer Catalyst)

The oxidation reaction of the present invention may be performed in the presence of a phase transfer catalyst. Alternatively, the oxidation reaction may be performed in the absence of the phase transfer catalyst. Whether or not to use the phase transfer catalyst can be appropriately determined by a person skilled in the art. Examples of the phase transfer catalyst include, but are not limited to, the following: quaternary ammonium salts (e.g., tetrabutylammonium chloride, tetrabutylammonium bromide, tetrabutylammonium iodide, tetrabutylammonium hydrogen sulfate, benzyltrimethylammonium chloride, benzyltrimethylammonium bromide, octyltrimethylammonium chloride, octyltrimethylammonium bromide, trioctylmethylammonium chloride, trioctylmethylammonium bromide, benzyllauryldimethylammonium chloride (benzyldodecyldimethylammonium chloride), benzyllauryldimethylammonium bromide (benzyldodecyldimethylammonium bromide), myristyltrimethylammonium chloride (tetradecyltrimethylammonium chloride), myristyltrimethylammonium bromide (tetradecyltrimethylammonium bromide), benzyldimethylstearylammonium chloride (benzyloctadecyldimethylammonium chloride), benzyldimethylstearylammonium bromide (benzyloctadecyldimethylammonium bromide), etc.), quaternary phosphonium salts (tetrabutylphosphonium bromide, tetraoctylphosphonium bromide, tetraphenylphosphonium bromide, etc.) and crown ethers (e.g., 12-crown-4,15-crown-5 and 18-crown-6). From the viewpoint of yield, suppression of by-products, economic efficiency, etc., preferred examples of the phase transfer catalyst include tetrabutylammonium chloride, tetrabutylammonium bromide and tetrabutylammonium hydrogen sulfate, and more preferably tetrabutylammonium hydrogen sulfate. Tetrabutylammonium hydrogen sulfate may be abbreviated as TBAHS.

The phase transfer catalyst may be used singly or in a combination of two or more kinds thereof in any ratio. The form of the phase transfer catalyst may be any form as long as the reaction proceeds. The form of the phase transfer catalyst can be appropriately selected by a person skilled in the art. The amount of the phase transfer catalyst used may be any amount as long as the reaction proceeds. The amount of the phase transfer catalyst used may be appropriately adjusted by a person skilled in the art. However, from the viewpoint of yield, suppression of by-products, economic efficiency, etc., in one embodiment, the amount of the phase transfer catalyst used is, for example, 0 (zero) to 0.5 mol, more than 0 (zero) and 0.5 mol or less, 0.005 to 0.5 mol, 0.01 to 0.5 mol, or 0.05 to 0.5 mol, and preferably 0 (zero) to 0.2 mol, more than 0 (zero) and 0.2 mol or less, 0.005 to 0.2 mol, 0.01 to 0.2 mol, or 0.05 to 0.2 mol, based on 1 mol of the compound of the formula (4) (raw material).

### (Reaction Solvent)

From the viewpoint of allowing the reaction to smoothly proceed, the oxidation reaction of the present invention is preferably performed in the presence of a solvent. The reaction solvent may be any solvent as long as the reaction proceeds. The reaction solvent may be a carboxylic acid or an organic solvent excluding carboxylic acids. In either case, the reaction solvent may be in the presence of a water solvent.

In one embodiment, examples of the reaction solvent include, but are not limited to, the following: aromatic hydrocarbon derivatives (e.g., benzenes optionally substituted with 1 to 3 (preferably 1 or 2) substituents selected from (C1-C4)alkyl (preferably (C1-C3)alkyl, and more preferably (C1-C2)alkyl) and a chlorine atom, specifically, e.g., benzene, toluene, xylene, chlorobenzene, dichlorobenzene and trichlorobenzene. Specific examples of the aromatic hydrocarbon derivative may include nitrobenzene), halogenated aliphatic hydrocarbons (e.g. (C1-C4)alkane optionally substituted with 1 to 10 halogen atoms (preferably chlorine atoms), preferably (C1-C2)alkane optionally substituted with 1 to 6 chlorine atoms, specifically, e.g., dichloromethane, 1,2-dichloroethane (EDC), and chloroform), alcohols (e.g., (C1-C6)alcohol, specifically, e.g., methanol, ethanol, propanol, 2-propanol, butanol, sec-butanol, isobutanol, tert-butanol, pentanol, sec-amyl alcohol, 3-pentanol, 2-methyl-1-butanol, isoamyl alcohol, tert-amyl alcohol and hexanol. The alcohols are preferably (C1-C5)alcohols, and more preferably (C1-C4)alcohols, and specific examples thereof include suitable examples of the above examples. Examples of the alcohols may include cyclohexanol.), nitriles (e.g., (C2-C5)alkane nitrile, preferably (C2-C3)alkane nitrile, specifically, e.g., acetonitrile, propionitrile, butyronitrile, isobutyronitrile, succinonitrile, and preferably acetonitrile. Herein, the C2 alkane nitrile is acetonitrile. Examples of the nitriles may include benzonitrile.), carboxylic acids (acetic acid, propionic acid, trifluoroacetic acid, dichloroacetic acid and trichloroacetic acid), carboxylic acid esters (e.g., (C1-C4)alkyl (C2-C6) carboxylate, preferably (C1-C4)alkyl (C2-C3)carboxylate, specifically, e.g., methyl acetate, ethyl acetate, propyl acetate, isopropyl acetate, butyl acetate and isomers thereof, and pentyl acetate and isomers thereof (in the present invention, the “isomer of butyl acetate” is an equivalent of “butyl acetate” and the “isomer of pentyl acetate” is an equivalent of “pentyl acetate”)), ethers (e.g., tetrahydrofuran (THF), 2-methyltetrahydrofuran (2-MeTHF), 1,4-dioxane, diisopropyl ether, dibutyl ether, di-tert-butyl ether, cyclopentyl methyl ether (CPME), methyl-tert-butyl ether, 1,2-dimethoxyethane (DME) and diglyme), ketones (e.g., acetone, methyl ethyl ketone (MEK), methyl isopropyl ketone (MIPK) and methyl isobutyl ketone (MIBK)), amides (e.g., N,N-di((C1-C4)alkyl) (C1-C4)alkanamide, specifically, e.g., N,N-dimethylformamide (DMF) and N,N-dimethylacetamide (DMAC). Examples of the amides may include N-methylpyrrolidone (NMP).), ureas (e.g., N,N′-dimethylimidazolidinone (DMI) and tetramethylurea), sulfones (e.g., sulfolane), water, and any combination thereof in any ratio.

2-propanol is also referred to as isopropyl alcohol or isopropanol.
tert-butanol is also referred to as tert-butyl alcohol.

From the viewpoint of yield, suppression of by-products, economic efficiency, etc., preferred examples of the reaction solvent include alcohols, nitriles, carboxylic acids, carboxylic acid esters, amides, water, and any combination thereof in any ratio.

More preferred examples of the reaction solvent include alcohols, nitriles, carboxylic acids, amides, water, and any combination thereof in any ratio.

Still more preferred examples of the reaction solvent include alcohols, nitriles, carboxylic acids, water, and any combination thereof in any ratio.

From the same viewpoint as above, preferred specific examples of the reaction solvent include methanol, ethanol, propanol, 2-propanol, butanol, sec-butanol, isobutanol, tert-butanol, pentanol, sec-amyl alcohol, 3-pentanol, 2-methyl-1-butanol, isoamyl alcohol, tert-amyl alcohol, acetonitrile, methyl acetate, ethyl acetate, propyl acetate, isopropyl acetate, butyl acetate and isomers thereof, pentyl acetate and isomers thereof, acetic acid, propionic acid, trifluoroacetic acid, dichloroacetic acid, trichloroacetic acid, N,N-dimethylformamide (DMF), N,N-dimethylacetamide (DMAC), water, and any combination thereof in any ratio.

More preferred specific examples of the reaction solvent include methanol, ethanol, propanol, 2-propanol, butanol, sec-butanol, isobutanol, tert-butanol, pentanol, sec-amyl alcohol, 3-pentanol, 2-methyl-1-butanol, isoamyl alcohol, tert-amyl alcohol, acetonitrile, acetic acid, dichloroacetic acid, N,N-dimethylformamide (DMF), N,N-dimethylacetamide (DMAC), water, and any combination thereof in any ratio.

Still more preferred specific examples of the reaction solvent include methanol, ethanol, propanol, 2-propanol, butanol, acetonitrile, acetic acid, dichloroacetic acid, N,N-dimethylformamide (DMF), water, and any combination thereof in any ratio.

Further preferred specific examples of the reaction solvent include methanol, acetonitrile, acetic acid, dichloroacetic acid, N,N-dimethylformamide (DMF), water, and any combination thereof in any ratio.

Further preferred specific examples of the reaction solvent include methanol, acetonitrile, acetic acid, dichloroacetic acid, water, and any combination thereof in any ratio.

In another embodiment, preferred reaction solvents are as described herein. For example, preferred reaction solvents are as described in [A-40] to [A-70] and [A-78] herein. Examples and specific examples thereof are as described herein. For example, all the processes described in [A-40] to [A-70] and [A-78] herein may be performed “in the presence of a water solvent”.

Examples of preferred organic solvents include organic solvents as defined herein by the following parameters.

### (Acceptor Number)

Herein, regarding the acceptor number, the following document can be referred to, for example, Christian Reichardt, “Solvents and Solvent Effects in Organic Chemistry”, 3rd, updated and enlarged edition, WILEY-VCH, 2003, p. 25-26. The definition of the acceptor number utilizing ³¹P-NMR chemical shift values is described in the above document, which is incorporated into the present invention by reference. Examples of the solvent having a specified acceptor number are described in the above document, which are incorporated into the present invention by reference.

### (Relative Permittivity)

Herein, for example, the following documents can be referred to regarding relative permittivity (also generally known as “dielectric constant”): Chemical Handbook (Kagaku Binran) (basic edition) “edited by The Chemical Society of Japan, Maruzen Company, Limited, 5th edition, 2004, p. I-770 to 777; and A. Maryott and Edgar R. Smith, National Bureau of Standards Circular 514, Table of Dielectric Constants of Pure Liquids, United States Department of Commerce, National Bureau of Standards, Aug. 10, 1951, which are incorporated herein by reference. Examples of the solvent having a specified value of relative permittivity are described in the above document, which are incorporated into the present invention by reference.

### (Polarity Parameter of Rohrschneider)

Regarding the polarity parameter of Rohrschneider, for example, the following website can be referred to: https://www.shodex.com/ja/dc/06/0117.html, which is incorporated herein by reference. Examples of the solvent having a specified value of Rohrschneider polarity parameter are described in the above document, which are incorporated into the present invention by reference.

Examples of the organic solvent excluding carboxylic acids are as described herein. When the organic solvent excluding carboxylic acids is used, examples of the amount thereof are as follows from the viewpoint of yield, suppression of by-products, economic efficiency, etc.: in one embodiment, the lower limit of the amount of the organic solvent excluding carboxylic acids used is more than 0 (zero) liter or 0.1 liters or more, preferably 0.2 liters or more, more preferably 0.3 liters or more or 0.4 liters or more, and still more preferably 0.5 liters or more or 0.8 liters or more, based on 1 mol of the compound of the formula (1). In one embodiment, the upper limit of the amount of the organic solvent excluding carboxylic acids used is 5 liters or less, preferably 3 liters or less, more preferably 2 liters or less, and still more preferably 1 liter or less, based on 1 mol of the compound of the formula (1). The range of the amount of the organic solvent excluding carboxylic acids used is, for example, any appropriate combination of the upper and lower limits described above. For example, the combination of the upper and lower limits is as follows, but is not limited thereto: from the same viewpoint as the above, in one embodiment, the amount of the organic solvent excluding carboxylic acids used is, for example, 0.3 liters or more and 3 liters or less, and preferably 0.5 liters or more and 2 liters or less, based on 1 mol of the compound of the formula (1) (raw material).

In any case, the solvent may be in a single layer or may be separated into two layers as long as the reaction proceeds. Meanwhile, the present invention was considered after completion of the present invention, and it was also found that when a carboxylic acid and a specific organic solvent are used, preferable conditions (reaction systems) are obtained in the present invention from the viewpoint of solubility, affinity between the organic solvent and the water solvent, etc.

In addition, in the present invention, the use of the carboxylic acid was found to make it possible to select appropriate conditions depending on the purpose and the situation in industrial implementation. This is an advantageous effect of the present invention.

The “reaction solvents” are all “organic solvents excluding carboxylic acids”, “carboxylic acids used as solvents”, and “water solvents” used in the reaction. The organic solvent and the water solvent used in the working-up (e.g., isolation and purification) after the reaction are not included in the “reaction solvent”. The “organic solvent” used in the reaction includes the organic solvent in the raw material solution and that in the reactant solution. The “water solvent” used in the reaction includes the water in the raw material solution and that in the reactant solution (e.g., the water in an aqueous hydrogen peroxide solution).

The amount of the reaction solvent used is not particularly limited as long as the reaction system can be sufficiently stirred. However, from the viewpoint of yield, suppression of by-products, economic efficiency, etc., in one embodiment, the amount of the reaction solvent used is, for example, 0 (zero) to 10 L (liters), 0 (zero) to 5 L (liters), more than 0 (zero) and 10 L (liters) or less, or more than 0 (zero) and 5 L (liters) or less, preferably 0.2 to 10 L, 0.2 to 5 L, 0.2 to 3 L, or 0.2 to 2 L, more preferably 0.3 to 10 L, 0.3 to 5 L, 0.3 to 3 L, or 0.3 to 2 L, and still more preferably 0.4 to 10 L, 0.4 to 5 L, 0.4 to 3 L, or 0.4 to 2 L, based on 1 mol of the compound of the formula (1) (raw material). When a combination of two or more solvents is used, the ratio of the two or more solvents may be any ratio as long as the reaction proceeds.

### (Reaction Temperature)

The reaction temperature is not particularly limited as long as the effects of the present invention are exhibited. However, from the viewpoint of yield, suppression of by-products, economic efficiency, etc., in one embodiment, the lower limit of the reaction temperature is, for example, 10° C. or higher, preferably 20° C. or higher, 25° C. or higher, 35° C. or higher, above 35° C., 40° C. or higher, 45° C. or higher, or 50° C. or higher. The upper limit of the reaction temperature is, for example, 200° C. or lower, 150° C. or lower, or 100° C. or lower, preferably 80° C. or lower, more preferably 75° C. or lower, below 75° C., 70° C. or lower, below 70° C., 65° C. or lower, or 60° C. or lower, and still more preferably 55° C. or lower, below 55° C., 50° C. or lower, below 50° C., 45° C. or lower, 40° C. or lower, or 35° C. or lower. The range of the reaction temperature is, for example, any appropriate combination of the upper limit and the lower limit. For example, the combination of the upper limit and the lower limit is as follows, but is not limited thereto. From the same viewpoint as the above, in another embodiment, the reaction temperature is, for example, 10° C. or higher and 100° C. or lower, preferably 20° C. or higher and 100° C. or lower, more preferably above 35° C. and 100° C. or lower, still more preferably 40° C. or higher and 100° C. or lower, further preferably 45° C. or higher and 100° C. or lower, and further preferably 50° C. or higher and 100° C. or lower. From the same viewpoint as the above, in still another embodiment, the reaction temperature is, for example, 10° C. or higher and 80° C. or lower, preferably 20° C. or higher and 80° C. or lower, more preferably above 35° C. and 80° C. or lower, still more preferably 40° C. or higher and 80° C. or lower, further preferably 45° C. or higher and 80° C. or lower, and further preferably 50° C. or higher and 80° C. or lower. From the same viewpoint as the above, in yet another embodiment, the reaction temperature is, for example, 10° C. or higher and 60° C. or lower, preferably 20° C. or higher and 60° C. or lower, more preferably above 35° C. and 60° C. or lower, still more preferably 40° C. or higher and 60° C. or lower, further preferably 45° C. or higher and 60° C. or lower, and further preferably 50° C. or higher and 60° C. or lower. A lower reaction temperature is more preferable in terms of safety. A reaction temperature closer to room temperature (ordinary temperature) is more environmentally friendly and contributes to sustainability, but is not limited thereby.

### (Reaction Time)

The reaction time is not particularly limited as long as the effects of the present invention are exhibited. However, from the viewpoint of yield, suppression of by-products, economic efficiency, etc., in one embodiment, the lower limit of the reaction time is, for example, 1 hour or more, 1 hour 30 minutes or more or 2 hours or more, but is not limited thereto. In one embodiment, the upper limit of the reaction time is, for example, 48 hours or less or 36 hours or less, preferably 24 hours or less, 16 hours or less or 12 hours or less, but is not limited thereto. In another embodiment, the upper limit of the reaction time is, for example, 8 hours or less, 6 hours or less, 5 hours or less or 4 hours or less, but is not limited thereto. The range of the reaction time is, for example, any appropriate combination of the lower limit and the upper limit. The reaction time is, for example, 1 hour to 48 hours or 1 hour to 36 hours, and more preferably 1 hour to 24 hours, but is not limited thereto. However, the reaction time can be appropriately adjusted by a person skilled in the art depending on the purpose and the situation.

Hereinafter, the present invention will be described in more detail by Examples, but the present invention is not limited in any way by these Examples.

In the present description, the following instruments and conditions were used for the determination of physical properties and yields in Examples, Comparative Examples and Reference Examples. In addition, the products obtained in the present invention are known compounds, and were identified in the usual manner known to a person skilled in the art.

### (HPLC Analysis: High Performance Liquid Chromatography Analysis)

### (HPLC Analysis Conditions)

Instrument: LC 2010 Series manufactured by Shimadzu corporation or any equivalent thereto
Column: YMC-Pack, ODS-A, A-312 (150 mm×6.0 mm ID, S-5 μm, 120A)

### Eluent:

TABLE 1

| Time | Acetonitrile | 0.05% Phosphoric acid |
|---|---|---|
| (min) | (%) | aqueous solution (%) |
| 0 | 45 | 55 |
| 10 | 45 | 55 |
| 15 | 80 | 20 |
| 20 | 80 | 20 |

Flow rate: 1.0 ml/min

### Detection: UV 230 nm

Column temperature: 40° C.
Injection volume: 5 μL

The following documents can be referred to for the HPLC analysis method, as desired.
Document (a): “Shin Jikkenkagaku Koza 9 (A New Course in Experimental Chemistry 9) Bunsekikagaku II (Analytical Chemistry II)”, pages 86 to 112 (1977), edited by the Chemical Society of Japan, published by Shingo Iizumi, Maruzen Co., Ltd.
Document (b): “Jikkenkagaku Koza 20-1 (A Course in Experimental Chemistry 20-1) Bunseki Kagaku (Analytical Chemistry)”, 5th edition, pages 130 to 151 (2007), edited by the Chemical Society of Japan, published by Seishiro Murata, Maruzen Co., Ltd.
   (¹H-NMR: ¹H Nuclear Magnetic Resonance Spectrum)
Instrument: JEOL JMN-ECS-300 or JEOL JMN-Lambda-400 (manufactured by JEOL RESONANCE)
Solvent: CDCl₃ and/or DMSO-d_{ε}
Internal standard substance: tetramethylsilane (TMS) and others known to a person skilled in the art.

### (Yield and Purity)

Unless otherwise specified, the yield in the present invention can be calculated from the number of moles of the obtained target compound with respect to the number of moles of the raw material compound (starting compound).

That is, the term “yield” means “molar yield”.

Thus, the yield is represented by the following equation:

Yield %=the number of moles of the target compound obtained/(the number of moles of the starting compound)×100.

However, for example, in the evaluation of the reaction yield of the target product, the yield of impurities, the purity of the product, etc., HPLC area percentage analysis or GC area percentage analysis may be employed.

Herein, room temperature and ordinary temperature are from 10° C. to 35° C.

Herein, the term “overnight” means from 8 hours to 16 hours.

Herein, the procedure of “age/aged/aging” includes stirring a mixture by the usual manner known to a person skilled in the art.

In Examples herein, unless otherwise specified, “sulfuric acid” means concentrated sulfuric acid. Examples of the concentrated sulfuric acid include, but are not limited to, 98% sulfuric acid.

### EXAMPLES

### Example 1

### Production of 3-[(5-difluoromethoxy-1-methyl-3-trifluoromethylpyrazol-4-yl)methylsulfonyl]-4,5-dihydro-5,5-dimethylisoxazole (Compound 2-a)

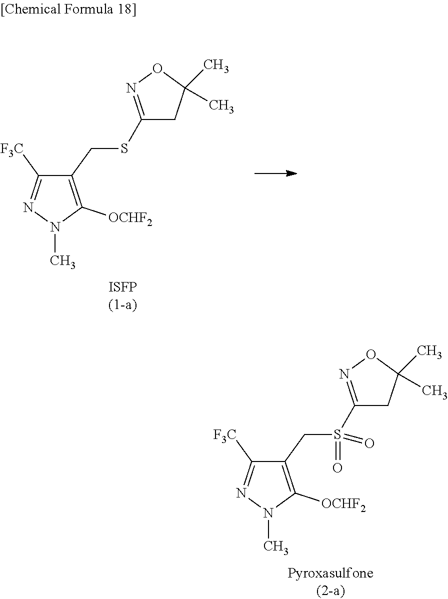 [SMILES: Cn1nc(C(F)(F)F)c(CSC2=NOC(C)(C)C2)c1OC(F)F ; Cn1nc(C(F)(F)F)c(CS(=O)(=O)C2=NOC(C)(C)C2)c1OC(F)F]

Under a nitrogen stream, the compound (1-a) (3.05 g, purity: 100%, 8.5 mmol, 100 mol %), 10.0 g (1.5 L/mol) of acetonitrile, acetic acid (1.53 g, 25.5 mmol, 300 mol %) and sodium tungstate dihydrate (0.084 g, 0.26 mmol, 3 mol %) were added to a reaction flask. A 35% aqueous hydrogen peroxide solution (2.48 g, 25.5 mmol, 300 mol %, containing 1.6 g (0.2 L/mol) of water) was added dropwise thereto at an internal temperature of 50° C. to 55° C. over 1 hour. The mixture was stirred at an internal temperature of 50° C. to 55° C. for 6 hours.

At this point of time, 3-[(5-difluoromethoxy-1-methyl-3-trifluoromethylpyrazol-4-yl)methylsulfinyl]-4,5-dihydro-5,5-dimethylisoxazole (Compound 3-a; SO derivative), which is a reaction intermediate, was 2.51% (HPLC area percentage; 230 nm). The mixture was homogeneous.

Acetonitrile was added to the reaction mixture. As a result of analysis by the HPLC external standard method, the target product (2-a) was obtained with a yield of 95.6%.

¹H-NMR value (CDCl₃/TMS δ (ppm)): 6.83 (1H, t, J=71.9 Hz), 4.60 (2H, s), 3.88 (3H, s), 3.11 (2H, s), 1.52 (6H, s)

### Example 2

### Production of 3-[(5-difluoromethoxy-1-methyl-3-trifluoromethylpyrazol-4-yl)methylsulfonyl]-4,5-dihydro-5,5-dimethylisoxazole (Compound 2-a)

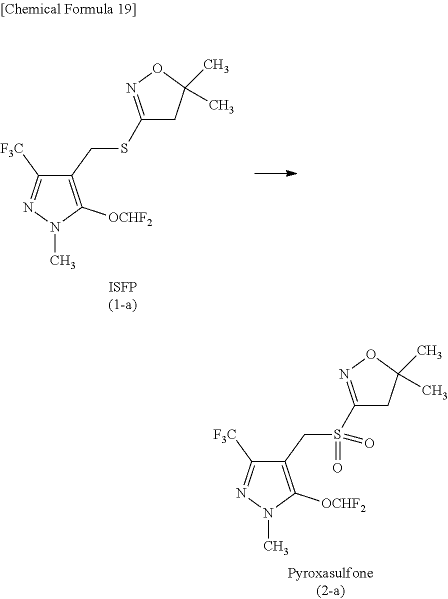 [SMILES: Cn1nc(C(F)(F)F)c(CSC2=NOC(C)(C)C2)c1OC(F)F ; Cn1nc(C(F)(F)F)c(CS(=O)(=O)C2=NOC(C)(C)C2)c1OC(F)F]

Under a nitrogen stream, the compound (1-a) (3.05 g, purity: 100%, 8.5 mmol, 100 mol %), 10.0 g (1.5 L/mol) of acetonitrile, acetic acid (0.26 g, 4.25 mmol, 50 mol %) and sodium tungstate dihydrate (0.084 g, 0.26 mmol, 3 mol %) were added to a reaction flask. A 35% aqueous hydrogen peroxide solution (2.48 g, 25.5 mmol, 300 mol %, containing 1.6 g (0.2 L/mol) of water) was added dropwise thereto at an internal temperature of 50° C. to 55° C. over 1 hour. The mixture was stirred at an internal temperature of 50° C. to 55° C. and aged for 12 hours.

At this point of time, 3-[(5-difluoromethoxy-1-methyl-3-trifluoromethylpyrazol-4-yl)methylsulfinyl]-4,5-dihydro-5,5-dimethylisoxazole (Compound 3-a; SO derivative), which is a reaction intermediate, was 1.90% (HPLC area percentage; 230 nm).

Acetonitrile was added to the reaction mixture. As a result of analysis by the HPLC external standard method, the target product (2-a) was obtained with a yield of 97.4%.

### Example 3

### Production of 3-[(5-difluoromethoxy-1-methyl-3-trifluoromethylpyrazol-4-yl)methylsulfonyl]-4,5-dihydro-5,5-dimethylisoxazole (Compound 2-a)

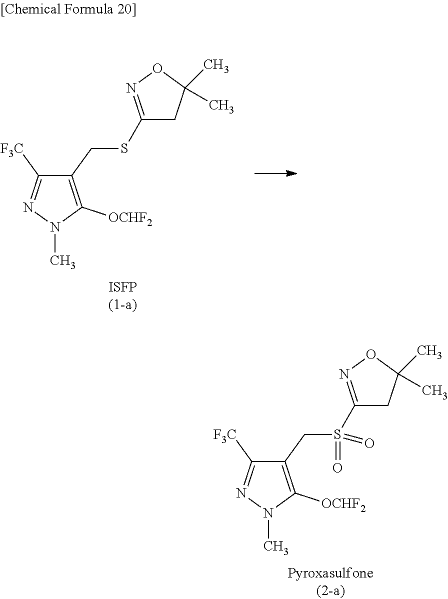 [SMILES: Cn1nc(C(F)(F)F)c(CSC2=NOC(C)(C)C2)c1OC(F)F ; Cn1nc(C(F)(F)F)c(CS(=O)(=O)C2=NOC(C)(C)C2)c1OC(F)F]

Under a nitrogen stream, the compound (1-a) (3.05 g, purity: 100%, 8.5 mmol, 100 mol %), 10.0 g (1.5 L/mol) of acetonitrile, acetic acid (0.051 g, 0.85 mmol, 10 mol %) and sodium tungstate dihydrate (0.084 g, 0.26 mmol, 3 mol %) were added to a reaction flask. A 35% aqueous hydrogen peroxide solution (2.48 g, 25.5 mmol, 300 mol %, containing 1.6 g (0.2 L/mol) of water) was added dropwise thereto at an internal temperature of 50° C. to 55° C. over 1 hour. The mixture was stirred at an internal temperature of 50° C. to 55° C. and aged for 12 hours.

At this point of time, 3-[(5-difluoromethoxy-1-methyl-3-trifluoromethylpyrazol-4-yl)methylsulfinyl]-4,5-dihydro-5,5-dimethylisoxazole (Compound 3-a; SO derivative), which is a reaction intermediate, was 2.87% (HPLC area percentage; 230 nm).

Acetonitrile was added to the reaction mixture. As a result of analysis by the HPLC external standard method, the target product (2-a) was obtained with a yield of 96.4%.

### Reference Example 1

### Reproduction Experiment of Example 9C in JP 2013-512201 A (Patent Document 3)

### Production of 3-[(5-difluoromethoxy-1-methyl-3-trifluoromethylpyrazol-4-yl)methylsulfonyl]-4,5-dihydro-5,5-dimethylisoxazole (Compound 2-a)

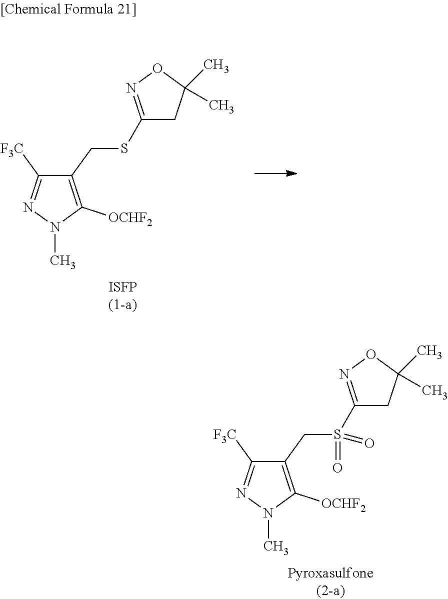 [SMILES: Cn1nc(C(F)(F)F)c(CSC2=NOC(C)(C)C2)c1OC(F)F ; Cn1nc(C(F)(F)F)c(CS(=O)(=O)C2=NOC(C)(C)C2)c1OC(F)F]

Under a nitrogen stream, 2.8 g (100 mol %) of the compound (1-a), 8.4 g (1.0 L/mol) of acetic acid and 80 mg (3 mol %) of sodium tungstate dihydrate were added to a reaction flask. To the mixture was added 2.2 g of a 30% aqueous hydrogen peroxide solution (250 mol %) dropwise at an internal temperature of 26° C. to 35° C. over 20 minutes, and the resulting mixture was aged for 16 hours while the internal temperature was maintained at 26° C. to 35° C.

At the point when the aging had been performed for 16 hours, 3-[(5-difluoromethoxy-1-methyl-3-trifluoromethylpyrazol-4-yl)methylsulfinyl]-4,5-dihydro-5,5-dimethylisoxazole (Compound 3-a; SO derivative), which is a reaction intermediate, remained 5.0% (HPLC area percentage).

To the reaction mixture was added 4 g of water, and the resulting mixture was aged at 10° C. for 1 hour, and then the precipitated crystals were separated by filtration.

The obtained crystals were successively washed with 20 ml of petroleum ether and 20 ml of water. The obtained crystals were analyzed by HPLC (area percentage; 230 nm). As a result, 3-[(5-difluoromethoxy-1-methyl-3-trifluoromethylpyrazol-4-yl)methylsulfinyl]-4,5-dihydro-5,5-dimethylisoxazole (Compound 3-a; SO derivative), which is a reaction intermediate, had an HPLC area percentage of 5.5%.

Reference Example 1 is a reproduction experiment of Example 9C in JP 2013-512201 A (Patent Document 3). In the production process described in JP2013-512201A (Patent Document 3), even after aging for 16 hours, the compound (3-a), which is a reaction intermediate, remained no less than 5.0%. In addition, even after purification, the ratio of the compound (3-a) did not decrease. It has been confirmed that it is difficult to purify the compound of the formula (2) by separating the compound of the formula (2) from the compound of the formula (3).

### Reference Example 2

### Reproduction Experiment of Example 5 in CN 111574511 A (Patent Document 5)

### Production of 3-[(5-difluoromethoxy-1-methyl-3-trifluoromethylpyrazol-4-yl)methylsulfonyl]-4,5-dihydro-5,5-dimethylisoxazole (Compound 2-a)

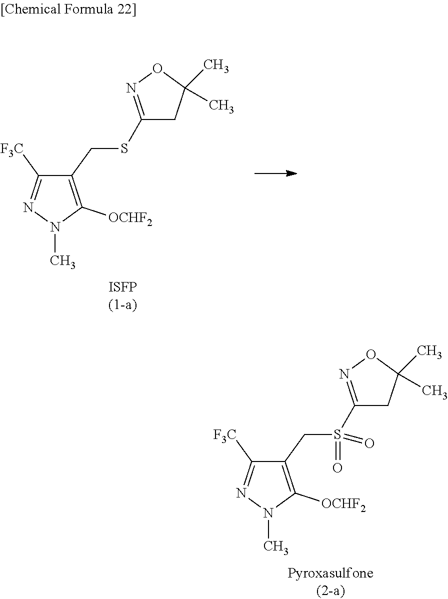 [SMILES: Cn1nc(C(F)(F)F)c(CSC2=NOC(C)(C)C2)c1OC(F)F ; Cn1nc(C(F)(F)F)c(CS(=O)(=O)C2=NOC(C)(C)C2)c1OC(F)F]

Under a nitrogen stream, the compound (1-a) (3.05 g, purity: 100%, 8.5 mmol, 100 mol %), acetic acid (13.4 g, 223 mmol, 2600 mol %, 1.5 L/mol), sulfuric acid (0.078 g, 0.765 mmol, 9 mol %) and sodium tungstate dihydrate (0.056 g, 0.170 mmol, 2 mol %) were added to a reaction flask. A 30% aqueous hydrogen peroxide solution (2.75 g, 24.2 mmol, 285 mol %, containing 1.9 g (0.23 L/mol) of water) was added dropwise thereto at an internal temperature (25° C. to 30° C.) over 1 hour. The mixture was stirred at room temperature (internal temperature of 25° C. to 30° C.) for 6 hours.

At this point of time, 3-[(5-difluoromethoxy-1-methyl-3-trifluoromethylpyrazol-4-yl)methylsulfinyl]-4,5-dihydro-5,5-dimethylisoxazole (Compound 3-a; SO derivative), which is a reaction intermediate, was 12.74% (HPLC area percentage; 230 nm).

Acetonitrile was added to the reaction mixture to form a homogeneous solution. As a result of analysis by the HPLC external standard method, the target product (2-a) was obtained with a yield of 79.9%.

Reference Example 2 is a reproduction experiment of Example 5 in CN 111574511 A (Patent Document 5). In the production process described in CN 111574511 A (Patent Document 5), the compound (3-a), which is a reaction intermediate, remained even though a large amount of carboxylic acid (acetic acid) was used.

### Example 4

### Production of 3-[(5-difluoromethoxy-1-methyl-3-trifluoromethylpyrazol-4-yl)methylsulfonyl]-4,5-dihydro-5,5-dimethylisoxazole (Compound 2-a)

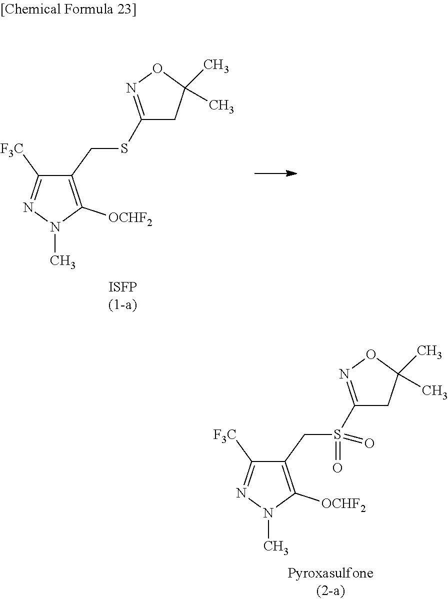 [SMILES: Cn1nc(C(F)(F)F)c(CSC2=NOC(C)(C)C2)c1OC(F)F ; Cn1nc(C(F)(F)F)c(CS(=O)(=O)C2=NOC(C)(C)C2)c1OC(F)F]

Under a nitrogen stream, the compound (1-a) (3.05 g, purity: 100%, 8.5 mmol, 100 mol %), acetic acid (13.4 g, 223 mmol, 2600 mol %, 1.5 L/mol), sulfuric acid (0.078 g, 0.765 mmol, 9 mol %) and sodium tungstate dihydrate (0.056 g, 0.170 mmol, 2 mol %) were added to a reaction flask. A 30% aqueous hydrogen peroxide solution (2.75 g, 24.2 mmol, 285 mol %, containing 1.9 g (0.23 L/mol) of water) was added dropwise thereto at an internal temperature of 71° C. over 1 hour. The mixture was stirred at 71° C. for 6 hours.

At this point of time, 3-[(5-difluoromethoxy-1-methyl-3-trifluoromethylpyrazol-4-yl)methylsulfinyl]-4,5-dihydro-5,5-dimethylisoxazole (Compound 3-a; SO derivative), which is a reaction intermediate, was 0% (HPLC area percentage; 230 nm).

Acetonitrile was added to the reaction mixture to form a homogeneous solution. As a result of analysis by the HPLC external standard method, the target product (2-a) was obtained with a yield of 88.0%.

### Example 5

Production of 3-[(5-difluoromethoxy-1-methyl-3-trifluoromethylpyrazol-4-yl)methylsulfonyl]-4,5-dihydro-5,5-dimethylisoxazole (Compound 2-a)

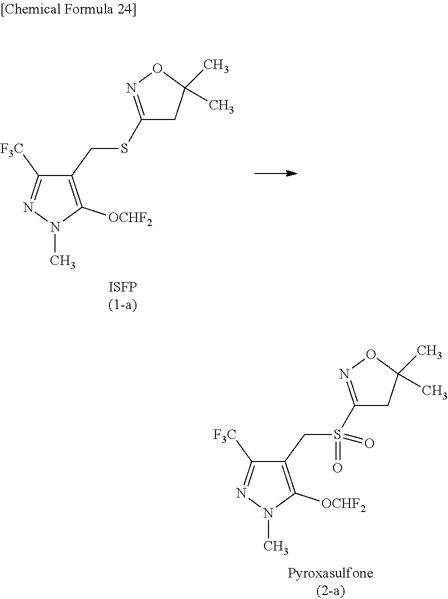 [SMILES: Cn1nc(C(F)(F)F)c(CSC2=NOC(C)(C)C2)c1OC(F)F ; Cn1nc(C(F)(F)F)c(CS(=O)(=O)C2=NOC(C)(C)C2)c1OC(F)F]

Under a nitrogen stream, the compound (1-a) (0.90 g, purity: 100%, 2.5 mmol, 100 mol %), acetic acid (3.90 g, 65.0 mmol, 2600 mol %, 1.5 L/mol), sodium tungstate dihydrate (0.0165 g, 0.050 mmol, 2 mol %), sulfuric acid (0.025 g, 0.25 mmol, 10 mol %) and a 35% aqueous hydrogen peroxide solution (0.69 g, 7.13 mmol, 285 mol %, containing 0.45 g (0.18 L/mol) of water) were added to a reaction flask. After the mixture was stirred at an internal temperature of 50° C. to 55° C. and aged for 2 hours, crystals were precipitated, and the mixture became a suspension. The suspension was aged at an internal temperature of 50° C. to 55° C. for another 2 hours.

At this point of time, 3-[(5-difluoromethoxy-1-methyl-3-trifluoromethylpyrazol-4-yl)methylsulfinyl]-4,5-dihydro-5,5-dimethylisoxazole (Compound 3-a; SO derivative), which is a reaction intermediate, was 0.4% (HPLC area percentage; 230 nm).

Acetonitrile was added to the reaction mixture to form a homogeneous solution. As a result of analysis by the HPLC external standard method, the target product (2-a) was obtained with a yield of 89.6%.

### Example 6

### Production of 3-[(5-difluoromethoxy-1-methyl-3-trifluoromethylpyrazol-4-yl)methylsulfonyl]-4,5-dihydro-5,5-dimethylisoxazole (Compound 2-a)

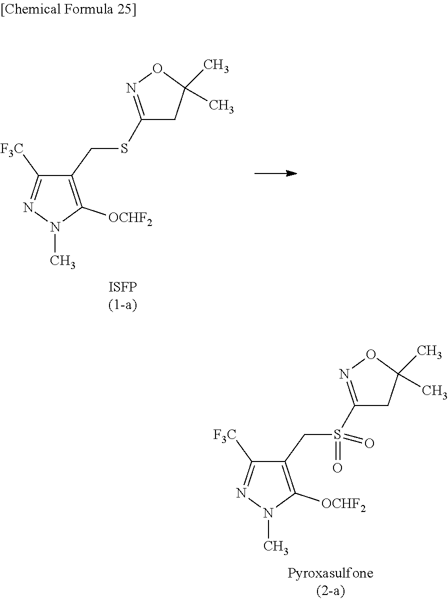 [SMILES: Cn1nc(C(F)(F)F)c(CSC2=NOC(C)(C)C2)c1OC(F)F ; Cn1nc(C(F)(F)F)c(CS(=O)(=O)C2=NOC(C)(C)C2)c1OC(F)F]

Under a nitrogen stream, the compound (1-a) (0.90 g, purity: 100%, 2.5 mmol, 100 mol %), acetic acid (2.69 g, 44.8 mmol, 1790 mol %, 1.0 L/mol) and sodium tungstate dihydrate (0.025 g, 0.075 mmol, 3 mol %) were added to a reaction flask. A 30% aqueous hydrogen peroxide solution (0.71 g, 6.25 mmol, 250 mol %, containing 0.50 g (0.2 L/mol) of water) was added dropwise thereto at an internal temperature of 50° C. to 55° C. over 20 minutes. After the mixture was stirred at an internal temperature of 50° C. to 55° C. and aged for 2 hours, crystals were precipitated, and the mixture became a suspension. The suspension was aged at an internal temperature of 50° C. to 55° C. for another 2 hours.

At this point of time, 3-[(5-difluoromethoxy-1-methyl-3-trifluoromethylpyrazol-4-yl)methylsulfinyl]-4,5-dihydro-5,5-dimethylisoxazole (Compound 3-a; SO derivative), which is a reaction intermediate, was 1.1% (HPLC area percentage; 230 nm).

Acetonitrile was added to the reaction mixture to form a homogeneous solution. As a result of analysis by the HPLC external standard method, the target product (2-a) was obtained with a yield of 90.0%.

### Example 7

### Production of 3-[(5-difluoromethoxy-1-methyl-3-trifluoromethylpyrazol-4-yl)methylsulfonyl]-4,5-dihydro-5,5-dimethylisoxazole (Compound 2-a)

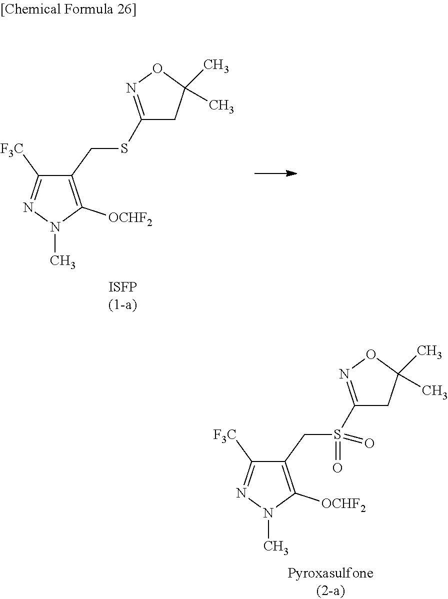 [SMILES: Cn1nc(C(F)(F)F)c(CSC2=NOC(C)(C)C2)c1OC(F)F ; Cn1nc(C(F)(F)F)c(CS(=O)(=O)C2=NOC(C)(C)C2)c1OC(F)F]

Under a nitrogen stream, the compound (1-a) (3.05 g, purity: 100%, 8.5 mmol, 100 mol %), 10.1 g (1.5 L/mol) of methanol, acetic acid (1.53 g, 25.5 mmol, 300 mol %) and sodium tungstate dihydrate (0.084 g, 0.26 mmol, 3 mol %) were added to a reaction flask, and the mixture was heated to an internal temperature of 50° C. to 55° C. A 35% aqueous hydrogen peroxide solution (2.48 g, 25.5 mmol, 300 mol %, containing 1.6 g (0.2 L/mol) of water) was added dropwise thereto at an internal temperature of 50° C. to 55° C. over 1 hour. The mixture was stirred at an internal temperature of 50° C. to 55° C. and aged for 9 hours.

At this point of time, 3-[(5-difluoromethoxy-1-methyl-3-trifluoromethylpyrazol-4-yl)methylsulfinyl]-4,5-dihydro-5,5-dimethylisoxazole (Compound 3-a; SO derivative), which is a reaction intermediate, was 2.15% (HPLC area percentage; 230 nm).

Acetonitrile was added to the reaction mixture to form a homogeneous solution. As a result of analysis by the HPLC external standard method, the target product (2-a) was obtained with a yield of 94.1%.

### Example 8

### Production of 3-[(5-difluoromethoxy-1-methyl-3-trifluoromethylpyrazol-4-yl)methylsulfonyl]-4,5-dihydro-5,5-dimethylisoxazole (Compound 2-a)

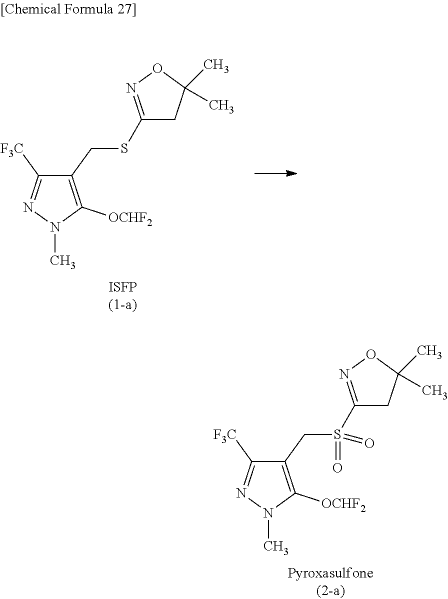 [SMILES: Cn1nc(C(F)(F)F)c(CSC2=NOC(C)(C)C2)c1OC(F)F ; Cn1nc(C(F)(F)F)c(CS(=O)(=O)C2=NOC(C)(C)C2)c1OC(F)F]

Under a nitrogen stream, the compound (1-a) (3.05 g, purity: 100%, 8.5 mmol, 100 mol %), 6.7 g of acetonitrile (1.0 L/mol), acetic acid (4.44 g, 74.0 mmol, 870 mol %, 0.5 L/mol) and sodium tungstate dihydrate (0.084 g, 0.26 mmol, 3 mol %) were added to a reaction flask, and the mixture was heated to an internal temperature of 50° C. to 55° C. A 35% aqueous hydrogen peroxide solution (2.48 g, 25.5 mmol, 300 mol %, containing 1.6 g (0.2 L/mol) of water) was added dropwise thereto at an internal temperature of 50° C. to 55° C. over 1 hour. The mixture was stirred at an internal temperature of 50° C. to 55° C. and aged for 5 hours. The mixture was homogeneous.

At this point of time, 3-[(5-difluoromethoxy-1-methyl-3-trifluoromethylpyrazol-4-yl)methylsulfinyl]-4,5-dihydro-5,5-dimethylisoxazole (Compound 3-a; SO derivative), which is a reaction intermediate, was 0.22% (HPLC area percentage; 230 nm).

Acetonitrile was added to the reaction mixture. As a result of analysis by the HPLC external standard method, the target product (2-a) was obtained with a yield of 91.3%.

### Example 9

### Production of 3-[(5-difluoromethoxy-1-methyl-3-trifluoromethylpyrazol-4-yl)methylsulfonyl]-4,5-dihydro-5,5-dimethylisoxazole (Compound 2-a)

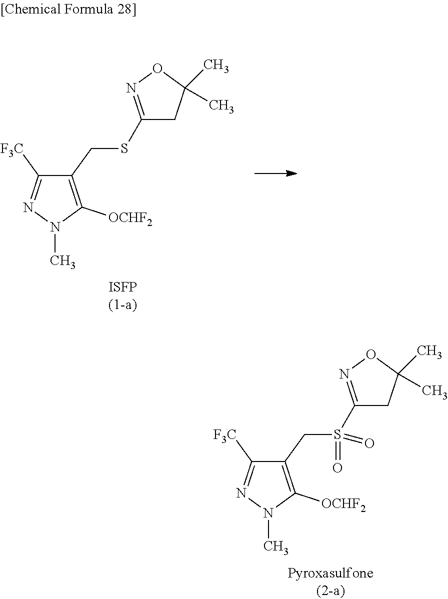 [SMILES: Cn1nc(C(F)(F)F)c(CSC2=NOC(C)(C)C2)c1OC(F)F ; Cn1nc(C(F)(F)F)c(CS(=O)(=O)C2=NOC(C)(C)C2)c1OC(F)F]

Under a nitrogen stream, the compound (1-a) (3.05 g, purity: 100%, 8.5 mmol, 100 mol %), 6.7 g of acetonitrile (1.0 L/moi), acetic acid (4.44 g, 74.0 mmol, 870 mol %, 0.5 L/mol), sulfuric acid (0.085 g, 0.85 mmol, 10 mol %) and sodium tungstate dihydrate (0.084 g, 0.26 mmol, 3 mol %) were added to a reaction flask, and the mixture was heated to an internal temperature of 50° C. to 55° C. A 35% aqueous hydrogen peroxide solution (2.48 g, 25.5 mmol, 300 mol %, containing 1.6 g (0.2 L/mol) of water) was added dropwise thereto at an internal temperature of 50° C. to 55° C. over 1 hour.

The mixture was stirred at an internal temperature of 50° C. to 55° C. and aged for 3 hours.

At this point of time, 3-[(5-difluoromethoxy-1-methyl-3-trifluoromethylpyrazol-4-yl)methylsulfinyl]-4,5-dihydro-5,5-dimethylisoxazole (Compound 3-a; SO derivative), which is a reaction intermediate, was 0% (HPLC area percentage; 230 nm).

Acetonitrile was added to the reaction mixture. As a result of analysis by the HPLC external standard method, the target product (2-a) was obtained with a yield of 95.5%.

### Example 10

### Production of 3-[(5-difluoromethoxy-1-methyl-3-trifluoromethylpyrazol-4-yl)methylsulfonyl]-4,5-dihydro-5,5-dimethylisoxazole (Compound 2-a)

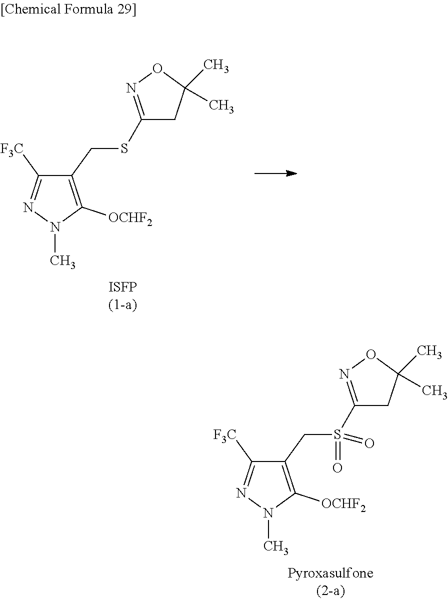 [SMILES: Cn1nc(C(F)(F)F)c(CSC2=NOC(C)(C)C2)c1OC(F)F ; Cn1nc(C(F)(F)F)c(CS(=O)(=O)C2=NOC(C)(C)C2)c1OC(F)F]

Under a nitrogen stream, the compound (1-a) (3.05 g, purity: 100%, 8.5 mmol, 100 mol %), 6.75 g (1.0 L/mol) of methanol, acetic acid (4.44 g, 74.0 mmol, 870 mol %, 0.5 L/mol) and sodium tungstate dihydrate (0.084 g, 0.26 mmol, 3 mol %) were added to a reaction flask, and the mixture was heated to an internal temperature of 50° C. to 55° C. A 35% aqueous hydrogen peroxide solution (2.48 g, 25.5 mmol, 300 mol %, containing 1.6 g (0.2 L/mol) of water) was added dropwise thereto at an internal temperature of 50° C. to 55° C. over 1 hour. The mixture was stirred at an internal temperature of 50° C. to 55° C. and aged for 5 hours.

At this point of time, 3-[(5-difluoromethoxy-1-methyl-3-trifluoromethylpyrazol-4-yl)methylsulfinyl]-4,5-dihydro-5,5-dimethylisoxazole (Compound 3-a; SO derivative), which is a reaction intermediate, was 1.98% (HPLC area percentage; 230 nm).

Acetonitrile was added to the reaction mixture to form a homogeneous solution. As a result of analysis by the HPLC external standard method, the target product (2-a) was obtained with a yield of 96.2%.

### Example 11

### Production of 3-[(5-difluoromethoxy-1-methyl-3-trifluoromethylpyrazol-4-yl)methylsulfonyl]-4,5-dihydro-5,5-dimethylisoxazole (Compound 2-a)

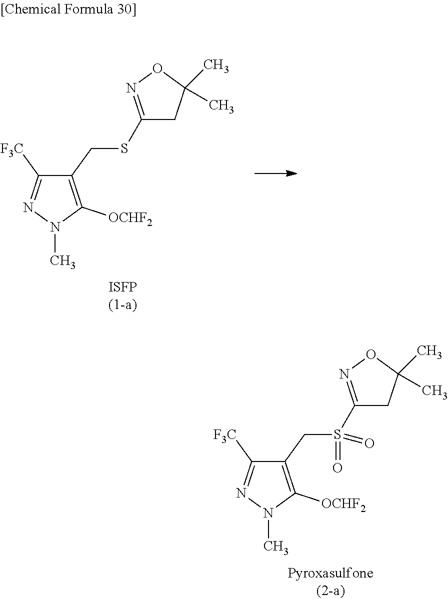 [SMILES: Cn1nc(C(F)(F)F)c(CSC2=NOC(C)(C)C2)c1OC(F)F ; Cn1nc(C(F)(F)F)c(CS(=O)(=O)C2=NOC(C)(C)C2)c1OC(F)F]

Under a nitrogen stream, the compound (1-a) (3.05 g, purity: 100%, 8.5 mmol, 100 mol %), acetic acid (17.7 g, 295 mmol, 3470 mol %, 2 L/mol) and sodium tungstate dihydrate (0.084 g, 0.26 mmol, 3 mol %) were added to a reaction flask. A 35% aqueous hydrogen peroxide solution (2.48 g, 25.5 mmol, 300 mol %, containing 1.6 g (0.2 L/mol) of water) was added dropwise thereto at an internal temperature of 25° C. to 30° C. over 1 hour. The mixture was stirred at an internal temperature of 25° C. to 30° C. and aged for 24 hours.

At this point of time, 3-[(5-difluoromethoxy-1-methyl-3-trifluoromethylpyrazol-4-yl)methylsulfinyl]-4,5-dihydro-5,5-dimethylisoxazole (Compound 3-a; SO derivative), which is a reaction intermediate, was 1.88% (HPLC area percentage; 230 nm).

Acetonitrile was added to the reaction mixture to form a homogeneous solution. As a result of analysis by the HPLC external standard method, the target product (2-a) was obtained with a yield of 93.0%.

### Example 12

### Production of 3-[(5-difluoromethoxy-1-methyl-3-trifluoromethylpyrazol-4-yl)methylsulfonyl]-4,5-dihydro-5,5-dimethylisoxazole (Compound 2-a)

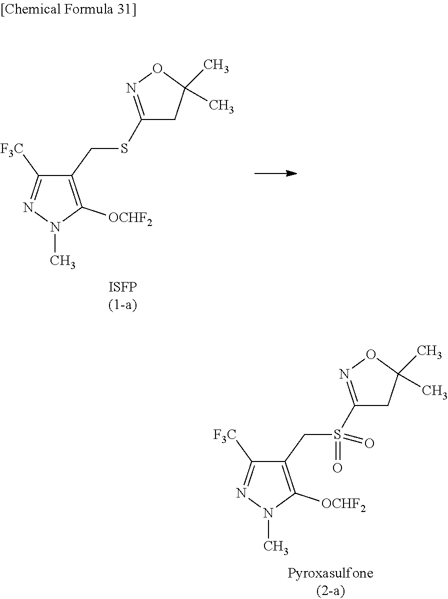 [SMILES: Cn1nc(C(F)(F)F)c(CSC2=NOC(C)(C)C2)c1OC(F)F ; Cn1nc(C(F)(F)F)c(CS(=O)(=O)C2=NOC(C)(C)C2)c1OC(F)F]

Under a nitrogen stream, the compound (1-a) (0.90 g, purity: 100%, 2.5 mmol, 100 mol %), acetic acid (3.90 g, 65.0 mmol, 2600 mol %, 1.5 L/mol), ammonium molybdate tetrahydrate (0.031 g, 0.025 mmol, 1 mol %), sulfuric acid (0.025 g, 0.25 mmol, 10 mol %) and a 35% aqueous hydrogen peroxide solution (0.73 g, 7.50 mmol, 300 mol %, containing 0.47 g (0.2 L/mol) of water) were added to a reaction flask. The mixture was stirred at an internal temperature of 50° C. to 55° C. and aged for 3 hours.

At this point of time, 3-[(5-difluoromethoxy-1-methyl-3-trifluoromethylpyrazol-4-yl)methylsulfinyl]-4,5-dihydro-5,5-dimethylisoxazole (Compound 3-a; SO derivative), which is a reaction intermediate, was 0.61% (HPLC area percentage; 230 nm).

Acetonitrile was added to the reaction mixture to form a homogeneous solution. As a result of analysis by the HPLC external standard method, the target product (2-a) was obtained with a yield of 94.3%.

### Example 13

### Production of 3-[(5-difluoromethoxy-1-methyl-3-trifluoromethylpyrazol-4-yl)methylsulfonyl]-4,5-dihydro-5,5-dimethylisoxazole (Compound 2-a)

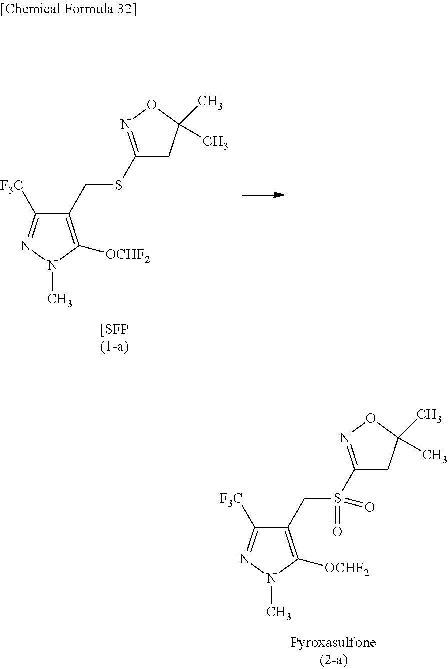 [SMILES: Cn1nc(C(F)(F)F)c(CSC2=NOC(C)(C)C2)c1OC(F)F ; Cn1nc(C(F)(F)F)c(CS(=O)(=O)C2=NOC(C)(C)C2)c1OC(F)F]

Under a nitrogen stream, the compound (1-a) (0.90 g, purity: 100%, 2.5 mmol, 100 mol %), acetic acid (3.90 g, 65.0 mmol, 2600 mol %, 1.5 L/mol), ammonium molybdate tetrahydrate (0.031 g, 0.025 mmol, 1 mol %) and a 35% aqueous hydrogen peroxide solution (0.73 g, 7.50 mmol, 300 mol %, containing 0.47 g (0.2 L/mol) of water) were added to a reaction flask. The mixture was stirred at an internal temperature of 50° C. to 55° C. and aged for 3 hours. At this point of time, 3-[(5-difluoromethoxy-1-methyl-3-trifluoromethylpyrazol-4-yl)methylsulfinyl]-4,5-dihydro-5,5-dimethylisoxazole (Compound 3-a; SO derivative), which is a reaction intermediate, was 0.95% (HPLC area percentage; 230 nm).

Acetonitrile was added to the reaction mixture to form a homogeneous solution. As a result of analysis by the HPLC external standard method, the target product (2-a) was obtained with a yield of 91.0%.

### Example 14

### Production of 3-[(5-difluoromethoxy-1-methyl-3-trifluoromethylpyrazol-4-yl)methylsulfonyl]-4,5-dihydro-5,5-dimethylisoxazole (Compound 2-a)

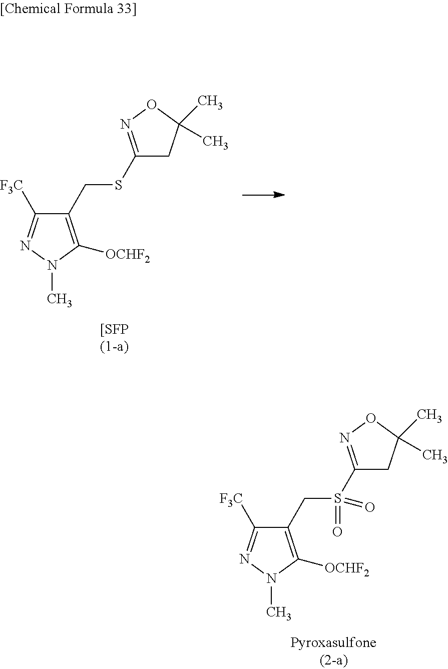 [SMILES: Cn1nc(C(F)(F)F)c(CSC2=NOC(C)(C)C2)c1OC(F)F ; Cn1nc(C(F)(F)F)c(CS(=O)(=O)C2=NOC(C)(C)C2)c1OC(F)F]

Under a nitrogen stream, the compound (1-a) (0.90 g, purity: 100%, 2.5 mmol, 100 mol %), acetic acid (2.61 g, 43.5 mmol, 1740 mol %, 1.0 L/mol), ammonium molybdate tetrahydrate (0.031 g, 0.025 mmol, 1 mol %) and a 35% aqueous hydrogen peroxide solution (0.73 g, 7.50 mmol, 300 mol %, containing 0.47 g (0.2 L/mol) of water) were added to a reaction flask. The mixture was stirred at an internal temperature of 50° C. to 55° C. and aged for 4 hours.

At this point of time, 3-[(5-difluoromethoxy-1-methyl-3-trifluoromethylpyrazol-4-yl)methylsulfinyl]-4,5-dihydro-5,5-dimethylisoxazole (Compound 3-a; SO derivative), which is a reaction intermediate, was 1.71% (HPLC area percentage; 230 nm).

Acetonitrile was added to the reaction mixture to form a homogeneous solution. As a result of analysis by the HPLC external standard method, the target product (2-a) was obtained with a yield of 95.6%.

### Example 15

### Production of 3-[(5-difluoromethoxy-1-methyl-3-trifluoromethylpyrazol-4-yl)methylsulfonyl]-4,5-dihydro-5,5-dimethylisoxazole (Compound 2-a)

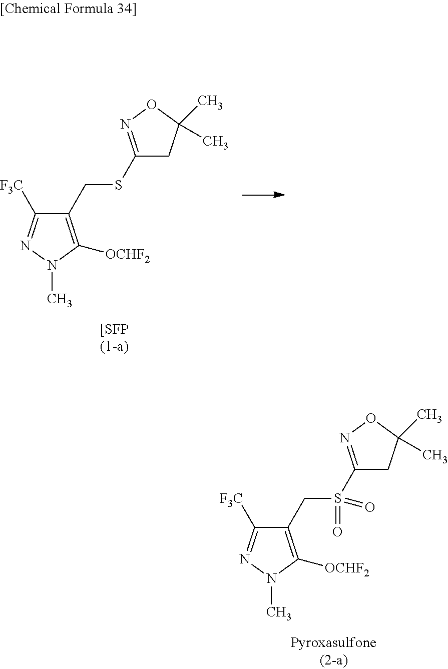 [SMILES: Cn1nc(C(F)(F)F)c(CSC2=NOC(C)(C)C2)c1OC(F)F ; Cn1nc(C(F)(F)F)c(CS(=O)(=O)C2=NOC(C)(C)C2)c1OC(F)F]

Under a nitrogen stream, the compound (1-a) (0.90 g, purity: 100%, 2.5 mmol, 100 mol %), acetic acid (0.075 g, 1.25 mmol, 50 mol %), ammonium molybdate tetrahydrate (0.031 g, 0.025 mmol, 1 mol %) and a 35% aqueous hydrogen peroxide solution (0.73 g, 7.50 mmol, 300 mol %, containing 0.47 g (0.2 L/mol) of water) were added to a reaction flask. The mixture was stirred at an internal temperature of 50° C. to 55° C. and aged for 3 hours.

At this point of time, 3-[(5-difluoromethoxy-1-methyl-3-trifluoromethylpyrazol-4-yl)methylsulfinyl]-4,5-dihydro-5,5-dimethylisoxazole (Compound 3-a; SO derivative), which is a reaction intermediate, was 0% (HPLC area percentage; 230 nm).

Acetonitrile was added to the reaction mixture. As a result of analysis by the HPLC external standard method, the target product (2-a) was obtained with a yield of 97.8%.

### Example 16

### Production of 3-[(5-difluoromethoxy-1-methyl-3-trifluoromethylpyrazol-4-yl)methylsulfonyl]-4,5-dihydro-5,5-dimethylisoxazole (Compound 2-a)

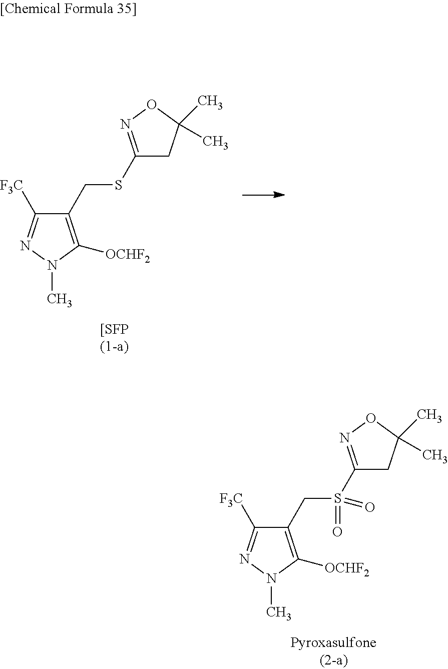 [SMILES: Cn1nc(C(F)(F)F)c(CSC2=NOC(C)(C)C2)c1OC(F)F ; Cn1nc(C(F)(F)F)c(CS(=O)(=O)C2=NOC(C)(C)C2)c1OC(F)F]

Under a nitrogen stream, the compound (1-a) (0.90 g, purity: 100%, 2.5 mmol, 100 mol %), acetonitrile (1.5 L/mol), acetic acid (0.45 g, 7.5 mmol, 300 mol %), ammonium molybdate tetrahydrate (0.031 g, 0.025 mmol, 1 mol %) and a 35% aqueous hydrogen peroxide solution (0.73 g, 7.50 mmol, 300 mol %, containing 0.47 g (0.2 L/mol) of water) were added to a reaction flask. The mixture was stirred at an internal temperature of 50° C. to 55° C. and aged for 6 hours.

At this point of time, 3-[(5-difluoromethoxy-1-methyl-3-trifluoromethylpyrazol-4-yl)methylsulfinyl]-4,5-dihydro-5,5-dimethylisoxazole (Compound 3-a; SO derivative), which is a reaction intermediate, was 0.45% (HPLC area percentage; 230 nm).

Acetonitrile was added to the reaction mixture. As a result of analysis by the HPLC external standard method, the target product (2-a) was obtained with a yield of 97.5%.

### Example 17

### Production of 3-[(5-difluoromethoxy-1-methyl-3-trifluoromethylpyrazol-4-yl)methylsulfonyl]-4,5-dihydro-5,5-dimethylisoxazole (Compound 2-a)

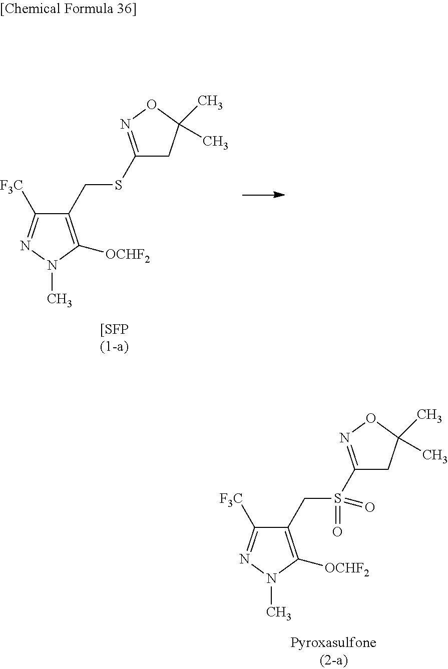 [SMILES: Cn1nc(C(F)(F)F)c(CSC2=NOC(C)(C)C2)c1OC(F)F ; Cn1nc(C(F)(F)F)c(CS(=O)(=O)C2=NOC(C)(C)C2)c1OC(F)F]

Under a nitrogen stream, the compound (1-a) (0.90 g, purity: 100%, 2.5 mmol, 100 mol %), acetonitrile (1.0 L/mol), acetic acid (1.31 g, 21.7 mmol, 870 mol %, 0.5 L/mol), ammonium molybdate tetrahydrate (0.031 g, 0.025 mmol, 1 mol %) and a 35% aqueous hydrogen peroxide solution (0.73 g, 7.50 mmol, 300 mol %, containing 0.47 g (0.2 L/mol) of water) were added to a reaction flask. The mixture was stirred at an internal temperature of 50° C. to 55° C. and aged for 6 hours.

At this point of time, 3-[(5-difluoromethoxy-1-methyl-3-trifluoromethylpyrazol-4-yl)methylsulfinyl]-4,5-dihydro-5,5-dimethylisoxazole (Compound 3-a; SO derivative), which is a reaction intermediate, was 0.16% (HPLC area percentage; 230 nm).

Acetonitrile was added to the reaction mixture. As a result of analysis by the HPLC external standard method, the target product (2-a) was obtained with a yield of 98.1%.

### Example 18

### Production of 3-[(5-difluoromethoxy-1-methyl-3-trifluoromethylpyrazol-4-yl)methylsulfonyl]-4,5-dihydro-5,5-dimethylisoxazole (Compound 2-a)

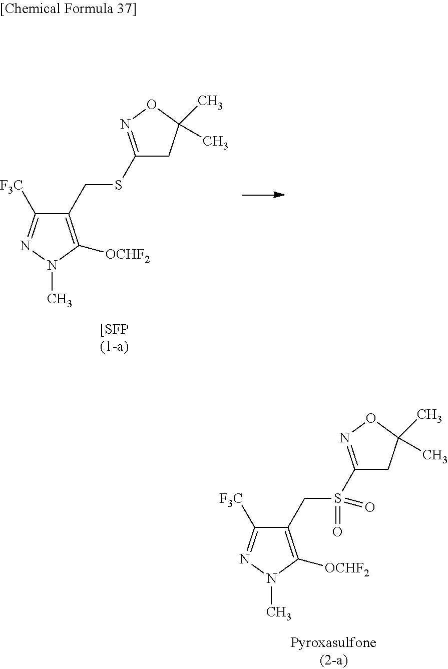 [SMILES: Cn1nc(C(F)(F)F)c(CSC2=NOC(C)(C)C2)c1OC(F)F ; Cn1nc(C(F)(F)F)c(CS(=O)(=O)C2=NOC(C)(C)C2)c1OC(F)F]

Under a nitrogen stream, the compound (1-a) (0.90 g, purity: 100%, 2.5 mmol, 100 mol %), methanol (1.0 L/mol), acetic acid (1.31 g, 21.7 mmol, 870 mol %, 0.5 L/mol), ammonium molybdate tetrahydrate (0.031 g, 0.025 mmol, 1 mol %) and a 35% aqueous hydrogen peroxide solution (0.73 g, 7.50 mmol, 300 mol %, containing 0.47 g (0.2 L/mol) of water) were added to a reaction flask. The mixture was stirred at an internal temperature of 50° C. to 55° C. and aged for 8 hours.

At this point of time, 3-[(5-difluoromethoxy-1-methyl-3-trifluoromethylpyrazol-4-yl)methylsulfinyl]-4,5-dihydro-5,5-dimethylisoxazole (Compound 3-a; SO derivative), which is a reaction intermediate, was 2.87% (HPLC area percentage; 230 nm).

Acetonitrile was added to the reaction mixture to form a homogeneous solution. As a result of analysis by the HPLC external standard method, the target product (2-a) was obtained with a yield of 93.3%.

### Example 19

### Production of 3-[(5-difluoromethoxy-1-methyl-3-trifluoromethylpyrazol-4-yl)methylsulfonyl]-4,5-dihydro-5,5-dimethylisoxazole (Compound 2-a)

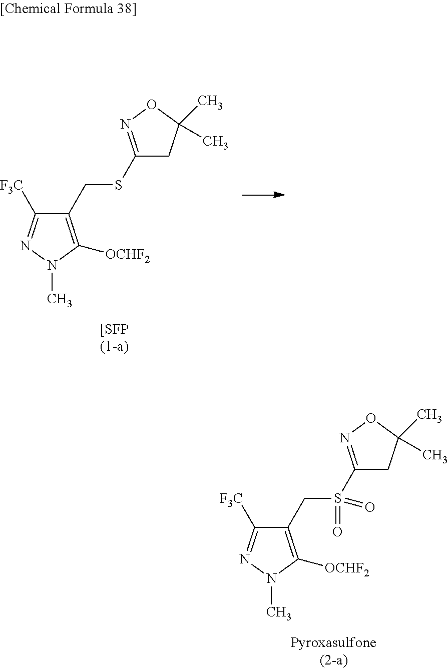 [SMILES: Cn1nc(C(F)(F)F)c(CSC2=NOC(C)(C)C2)c1OC(F)F ; Cn1nc(C(F)(F)F)c(CS(=O)(=O)C2=NOC(C)(C)C2)c1OC(F)F]

Under a nitrogen stream, the compound (1-a) (0.90 g, purity: 100%, 2.5 mmol, 100 mol %), acetonitrile (3.75 ml, 1.5 L/mol), trichloroacetic acid (1.23 g, 7.5 mmol, 300 mol %), sodium tungstate dihydrate (0.025 g, 0.075 mmol, 3 mol %) and a 35% aqueous hydrogen peroxide solution (0.73 g, 7.50 mmol, 300 mol %, containing 0.47 g (0.2 L/mol) of water) were added to a reaction flask. The mixture was stirred at an internal temperature of 50° C. to 55° C. and aged for 4 hours. The mixture was a homogeneous solution from the start to end of the reaction.

At this point of time, 3-[(5-difluoromethoxy-1-methyl-3-trifluoromethylpyrazol-4-yl)methylsulfinyl]-4,5-dihydro-5,5-dimethylisoxazole (Compound 3-a; SO derivative), which is a reaction intermediate, was 0% (HPLC area percentage; 230 nm).

Acetonitrile was added to the reaction mixture. As a result of analysis by the HPLC external standard method, the target product (2-a) was obtained with a yield of 89.9%.

### Example 20

### Production of 3-[(5-difluoromethoxy-1-methyl-3-trifluoromethylpyrazol-4-yl)methylsulfonyl]-4,5-dihydro-5,5-dimethylisoxazole (Compound 2-a)

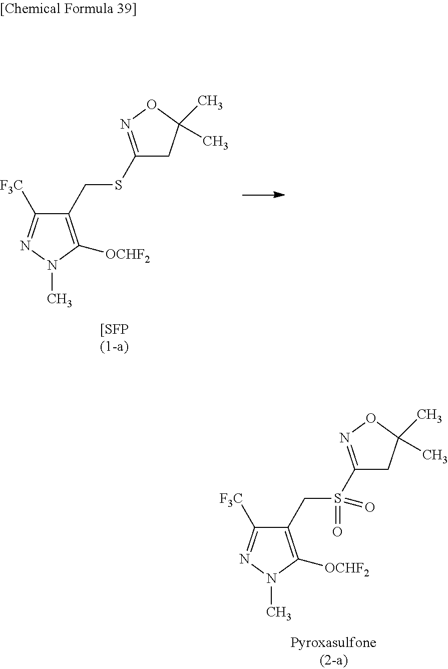 [SMILES: Cn1nc(C(F)(F)F)c(CSC2=NOC(C)(C)C2)c1OC(F)F ; Cn1nc(C(F)(F)F)c(CS(=O)(=O)C2=NOC(C)(C)C2)c1OC(F)F]

Under a nitrogen stream, the compound (1-a) (0.90 g, purity: 100%, 2.5 mmol, 100 mol %), dichloroacetic acid (5.85 g, 45.4 mmol, 1815 mol %, 1.5 L/mol), sodium tungstate dihydrate (0.025 g, 0.075 mmol, 3 mol %) and a 35% aqueous hydrogen peroxide solution (0.73 g, 7.50 mmol, 300 mol %, containing 0.47 g (0.2 L/mol) of water) were added to a reaction flask. The mixture was stirred at an internal temperature of 50° C. to 55° C. and aged for 2 hours. The mixture was a homogeneous solution from the start to end of the reaction.

At this point of time, 3-[(5-difluoromethoxy-1-methyl-3-trifluoromethylpyrazol-4-yl)methylsulfinyl]-4,5-dihydro-5,5-dimethylisoxazole (Compound 3-a; SO derivative), which is a reaction intermediate, was 0% (HPLC area percentage; 230 nm).

Acetonitrile was added to the reaction mixture. As a result of analysis by the HPLC external standard method, the target product (2-a) was obtained with a yield of 91.7%.

### Example 21

### Production of 3-[(5-difluoromethoxy-1-methyl-3-trifluoromethylpyrazol-4-yl)methylsulfonyl]-4,5-dihydro-5,5-dimethylisoxazole (Compound 2-a)

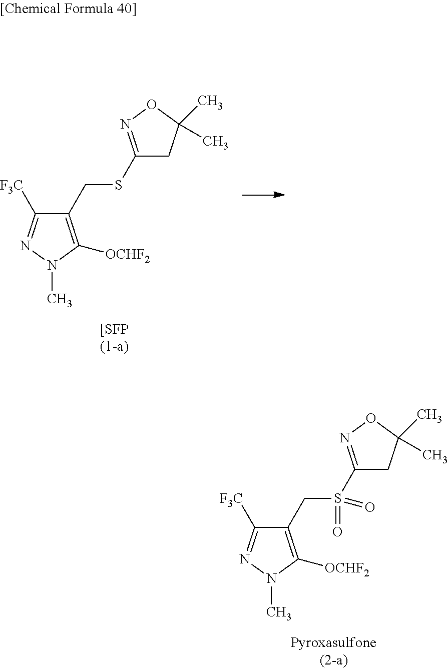 [SMILES: Cn1nc(C(F)(F)F)c(CSC2=NOC(C)(C)C2)c1OC(F)F ; Cn1nc(C(F)(F)F)c(CS(=O)(=O)C2=NOC(C)(C)C2)c1OC(F)F]

Under a nitrogen stream, the compound (1-a) (0.90 g, purity: 100%, 2.5 mmol, 100 mol %), dichloroacetic acid (5.85 g, 45.4 mmol, 1815 mol %, 1.5 L/mol), sodium tungstate dihydrate (0.025 g, 0.075 mmol, 3 mol %), sulfuric acid (0.025 g, 0.25 mmol, 10 mol %) and a 35% aqueous hydrogen peroxide solution (0.73 g, 7.50 mmol, 300 mol %, containing 0.47 g (0.2 L/mol) of water) were added to a reaction flask. The mixture was stirred at an internal temperature of 50° C. to 55° C. and aged for 2 hours. The mixture was a homogeneous solution from the start to end of the reaction.

At this point of time, 3-[(5-difluoromethoxy-1-methyl-3-trifluoromethylpyrazol-4-yl)methylsulfinyl]-4,5-dihydro-5,5-dimethylisoxazole (Compound 3-a; SO derivative), which is a reaction intermediate, was 0% (HPLC area percentage; 230 nm).

Acetonitrile was added to the reaction mixture. As a result of analysis by the HPLC external standard method, the target product (2-a) was obtained with a yield of 91.3%.

### Example 22

### Production of 3-[(5-difluoromethoxy-1-methyl-3-trifluoromethylpyrazol-4-yl)methylsulfonyl]-4,5-dihydro-5,5-dimethylisoxazole (Compound 2-a)

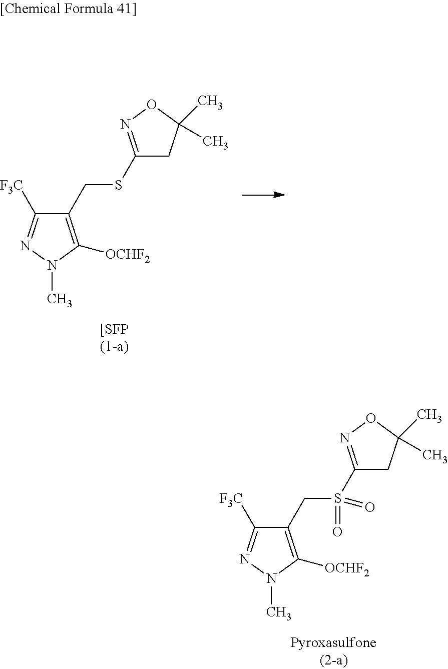 [SMILES: Cn1nc(C(F)(F)F)c(CSC2=NOC(C)(C)C2)c1OC(F)F ; Cn1nc(C(F)(F)F)c(CS(=O)(=O)C2=NOC(C)(C)C2)c1OC(F)F]

Under a nitrogen stream, the compound (1-a) (0.90 g, purity: 100%, 2.5 mmol, 100 mol %), acetonitrile (2.5 ml, 1.0 L/mol), dichloroacetic acid (1.95 g, 15.1 mmol, 605 mol %, 0.5 L/mol), sodium tungstate dihydrate (0.025 g, 0.075 mmol, 3 mol %) and a 35% aqueous hydrogen peroxide solution (0.73 g, 7.50 mmol, 300 mol %, containing 0.47 g (0.2 L/mol) of water) were added to a reaction flask. The mixture was stirred at an internal temperature of 50° C. to 55° C. and aged for 3.5 hours. The mixture was a homogeneous solution from the start to end of the reaction.

At this point of time, 3-[(5-difluoromethoxy-1-methyl-3-trifluoromethylpyrazol-4-yl)methylsulfinyl]-4,5-dihydro-5,5-dimethylisoxazole (Compound 3-a; SO derivative), which is a reaction intermediate, was 0% (HPLC area percentage; 230 nm).

Acetonitrile was added to the reaction mixture. As a result of analysis by the HPLC external standard method, the target product (2-a) was obtained with a yield of 91.7%.

### Example 23

### Production of 3-[(5-difluoromethoxy-1-methyl-3-trifluoromethylpyrazol-4-yl)methylsulfonyl]-4,5-dihydro-5,5-dimethylisoxazole (Compound 2-a)

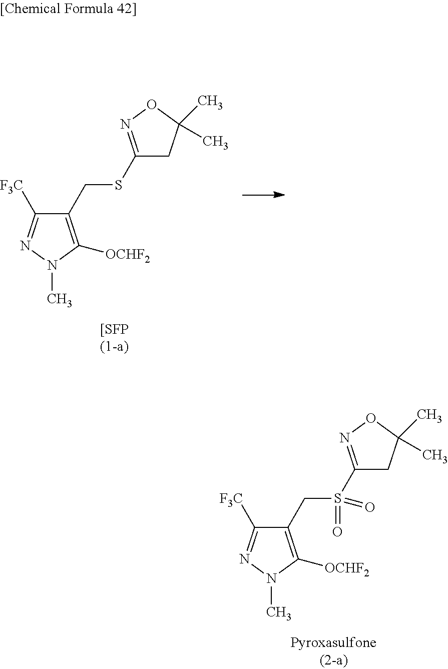 [SMILES: Cn1nc(C(F)(F)F)c(CSC2=NOC(C)(C)C2)c1OC(F)F ; Cn1nc(C(F)(F)F)c(CS(=O)(=O)C2=NOC(C)(C)C2)c1OC(F)F]

Under a nitrogen stream, the compound (1-a) (0.90 g, purity: 100%, 2.5 mmol, 100 mol %), dichloromethane (2.5 ml, 1.0 L/mol), dichloroacetic acid (1.95 g, 15.1 mmol, 605 mol %, 0.5 L/mol), sodium tungstate dihydrate (0.025 g, 0.075 mmol, 3 mol %) and a 35% aqueous hydrogen peroxide solution (0.73 g, 7.50 mmol, 300 mol %, containing 0.47 g (0.2 L/mol) of water) were added to a reaction flask. The mixture was stirred under heating reflux at an internal temperature of 41° C. and aged for 5.5 hours. The mixture was an emulsion from the start to end of the reaction.

At this point of time, 3-[(5-difluoromethoxy-1-methyl-3-trifluoromethylpyrazol-4-yl)methylsulfinyl]-4,5-dihydro-5,5-dimethylisoxazole (Compound 3-a; SO derivative), which is a reaction intermediate, was 0% (HPLC area percentage; 230 nm).

Acetonitrile was added to the reaction mixture to form a homogeneous solution. As a result of analysis by the HPLC external standard method, the target product (2-a) was obtained with a yield of 86.5%.

### Example 24

### Production of 3-[(5-difluoromethoxy-1-methyl-3-trifluoromethylpyrazol-4-yl)methylsulfonyl]-4,5-dihydro-5,5-dimethylisoxazole (Compound 2-a)

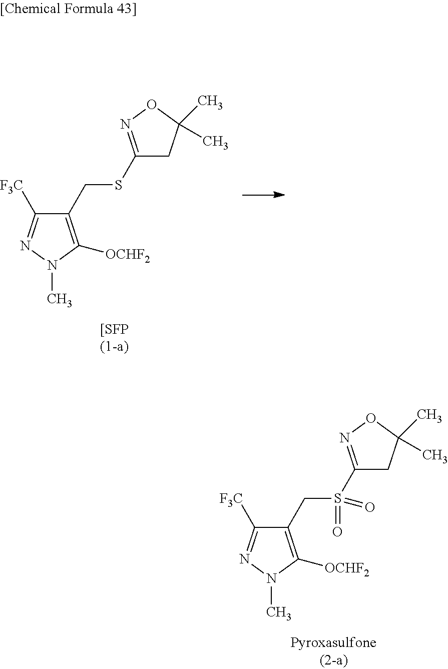 [SMILES: Cn1nc(C(F)(F)F)c(CSC2=NOC(C)(C)C2)c1OC(F)F ; Cn1nc(C(F)(F)F)c(CS(=O)(=O)C2=NOC(C)(C)C2)c1OC(F)F]

Under a nitrogen stream, the compound (1-a) (0.90 g, purity: 100%, 2.5 mmol, 100 mol %), dichloroacetic acid (5.85 g, 45.4 mmol, 1815 mol %, 1.5 L/mol), ammonium molybdate tetrahydrate (0.029 g, 0.025 mmol, 1 mol %) and a 35% aqueous hydrogen peroxide solution (0.73 g, 7.50 mmol, 300 mol %, containing 0.47 g (0.2 L/mol) of water) were added to a reaction flask. The mixture was stirred at an internal temperature of 50° C. to 55° C. and aged for 2 hours. The mixture was a homogeneous solution from the start to end of the reaction.

At this point of time, 3-[(5-difluoromethoxy-1-methyl-3-trifluoromethylpyrazol-4-yl)methylsulfinyl]-4,5-dihydro-5,5-dimethylisoxazole (Compound 3-a; SO derivative), which is a reaction intermediate, was 0% (HPLC area percentage; 230 nm).

Acetonitrile was added to the reaction mixture. As a result of analysis by the HPLC external standard method, the target product (2-a) was obtained with a yield of 89.3%.

All publications, patents, and patent applications described herein are hereby fully incorporated by reference in their entirety for the purpose of describing and disclosing the methods described in those publications, patents, and patent applications that may be used in connection with the description herein. To the extent necessary to understand or complete the disclosure of the present invention, all publications, patents, and patent applications described herein are expressly incorporated herein by reference to the same extent as if each were individually incorporated. All publications, patents, and patent applications discussed above and throughout this specification are provided solely for disclosure prior to the filing date of this application.

All processes described herein may be combined in any manner except where clearly contradicted by context. However, among the combinations of the processes described herein, combinations that contradict the contents thereof are excluded.

Any processes and reagents similar or equivalent to those described herein can be employed in the practice of the present invention. Accordingly, the present invention is not to be limited by the foregoing description, but is intended to be defined by the claims and their equivalents. Those equivalents fall within the scope of the present invention as defined by the appended claims.

1. A process for producing a compound of the formula (2), comprising:
a step of reacting a compound of the formula (1) with an oxidizing agent in the presence of a metal catalyst and the presence of a carboxylic acid,
wherein the reaction is performed at above 35° C., 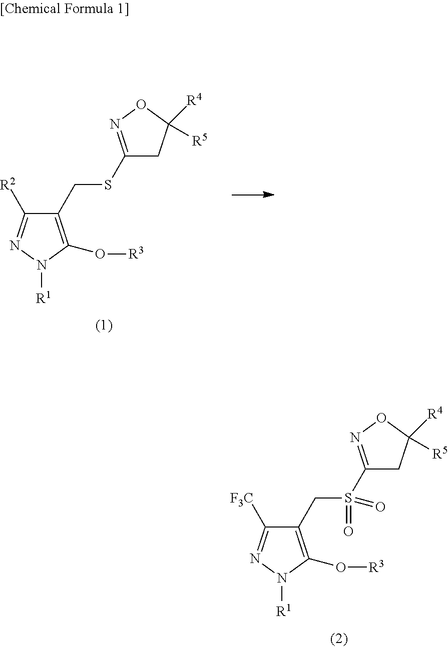
wherein in the formula (1) and (2),
R¹, R² and R³ are each independently a (C1-C6)alkyl optionally substituted with one or more substituents, a (C3-C6)cycloalkyl optionally substituted with one or more substituents, a (C2-C6)alkenyl optionally substituted with one or more substituents, a (C2-C6)alkynyl optionally substituted with one or more substituents, or a (C6-C10)aryl optionally substituted with one or more substituents, and
R⁴ and R⁵ are each independently a (C1-C6)alkyl optionally substituted with one or more substituents, a (C3-C6)cycloalkyl optionally substituted with one or more substituents, a (C2-C6)alkenyl optionally substituted with one or more substituents, a (C2-C6)alkynyl optionally substituted with one or more substituents, a (C1-C6)alkoxy optionally substituted with one or more substituents; or a (C6-C10)aryl optionally substituted with one or more substituents, or R⁴ and R⁵, together with the carbon atom to which they are attached, form a 3- to 12-membered carbocyclic ring, wherein the formed ring is optionally substituted with one or more substituents.

2. The process according to claim 1, wherein the reaction is performed at 40° C. or higher.

3. The process according to claim 1, wherein the reaction is performed at 45° C. or higher.

4. The process according to claim 1, wherein the reaction is performed at 60° C. or lower.

5. The process according to claim 1, wherein the reaction is performed at 55° C. or lower.

6. A process for producing a compound of the formula (2), comprising:
a step of reacting a compound of the formula (1) with an oxidizing agent in the presence of a metal catalyst and the presence of a carboxylic acid,
wherein an amount of the carboxylic acid is 18 mol or more based on 1 mol of the compound of the formula (1), 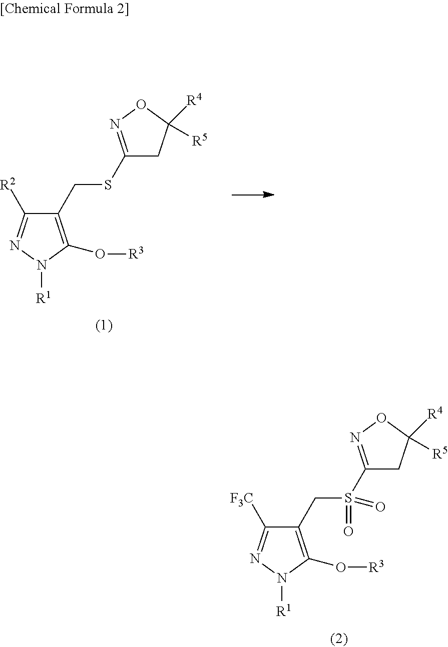
wherein in the formula (1) and (2),
R¹, R² and R³ are each independently a (C1-C6)alkyl optionally substituted with one or more substituents, a (C3-C6)cycloalkyl optionally substituted with one or more substituents, a (C2-C6)alkenyl optionally substituted with one or more substituents, a (C2-C6)alkynyl optionally substituted with one or more substituents, or a (C6-C10)aryl optionally substituted with one or more substituents, and
R⁴ and R⁵ are each independently a (C1-C6)alkyl optionally substituted with one or more substituents, a (C3-C6)cycloalkyl optionally substituted with one or more substituents, a (C2-C6)alkenyl optionally substituted with one or more substituents, a (C2-C6)alkynyl optionally substituted with one or more substituents, a (C1-C6)alkoxy optionally substituted with one or more substituents; or a (C6-C10)aryl optionally substituted with one or more substituents, or R⁴ and R⁵, together with the carbon atom to which they are attached, form a 3- to 12-membered carbocyclic ring, wherein the formed ring is optionally substituted with one or more substituents.

7. The process according to claim 6, wherein the amount of the carboxylic acid is 30 mol or more based on 1 mol of the compound of the formula (1).

8. The process according to claim 6, wherein the amount of the carboxylic acid is 35 mol or more based on 1 mol of the compound of the formula (1).

9. A process for producing a compound of the formula (2), comprising:
a step of reacting a compound of the formula (1) with an oxidizing agent in the presence of a metal catalyst and the presence of a carboxylic acid,
wherein the reaction is performed in the presence of an organic solvent excluding carboxylic acids, 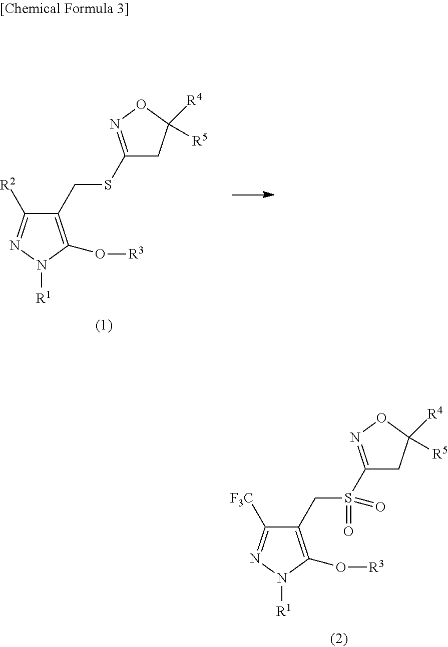
wherein in the formula (1) and (2),
R¹, R² and R³ are each independently a (C1-C6)alkyl optionally substituted with one or more substituents, a (C3-C6)cycloalkyl optionally substituted with one or more substituents, a (C2-C6)alkenyl optionally substituted with one or more substituents, a (C2-C6)alkynyl optionally substituted with one or more substituents, or a (C6-C10)aryl optionally substituted with one or more substituents, and
R⁴ and R⁵ are each independently a (C1-C6)alkyl optionally substituted with one or more substituents, a (C3-C6)cycloalkyl optionally substituted with one or more substituents, a (C2-C6)alkenyl optionally substituted with one or more substituents, a (C2-C6)alkynyl optionally substituted with one or more substituents, a (C1-C6)alkoxy optionally substituted with one or more substituents; or a (C6-C10)aryl optionally substituted with one or more substituents, or R⁴ and R⁵, together with the carbon atom to which they are attached, form a 3- to 12-membered carbocyclic ring, wherein the formed ring is optionally substituted with one or more substituents.

10. The process according to claim 9, wherein the organic solvent is selected from the group consisting of aromatic hydrocarbon derivatives, halogenated aliphatic hydrocarbons, alcohols, nitriles, carboxylic acid esters and amides.

11. The process according to claim 9, wherein the organic solvent is selected from the group consisting of halogenated aliphatic hydrocarbons, alcohols and nitriles.

12. The process according to claim 9, wherein the organic solvent is selected from the group consisting of (C1-C4)alkane optionally substituted with 1 to 10 halogen atoms, (C1-C6)alcohol and (C2-C5)alkane nitrile.

13. The process according to claim 9, wherein the organic solvent is selected from the group consisting of dichloromethane, methanol and acetonitrile.

14. The process according to claim 1, wherein the carboxylic acid is a carboxylic acid of the formula (a):
A-COOH (a) Chemical Formula 4
wherein A is hydrogen, a (C1-C6)alkyl optionally substituted with one or more substituents, a (C3-C6)cycloalkyl optionally substituted with one or more substituents, a (C2-C6)alkenyl optionally substituted with one or more substituents, or a (C2-C6)alkynyl optionally substituted with one or more substituents.

15. The process according to claim 1, wherein the carboxylic acid is acetic acid.

16. The process according to claim 1, wherein the carboxylic acid is dichloroacetic acid.

17. The process according to claim 1, wherein the carboxylic acid is trichloroacetic acid.

18. The process according to claim 1, wherein the metal catalyst is selected from the group consisting of a tungsten catalyst and a molybdenum catalyst.

19. The process according to claim 1, wherein the metal catalyst is a tungsten catalyst.

20. The process according to claim 1, wherein the metal catalyst is a molybdenum catalyst.

21. The process according to claim 1, wherein the oxidizing agent is hydrogen peroxide.

22. The process according to claim 1, wherein
R¹ is a (C1-C4)alkyl,
R² is a (C1-C4)perfluoroalkyl,
R³ is a (C1-C4)alkyl optionally substituted with 1 to 9 fluorine atoms, and
R⁴ and R⁵ are each independently a (C1-C4)alkyl.

23. The process according to claim 1, wherein
R¹ is methyl,
R² is trifluoromethyl,
R³ is difluoromethyl, and
R⁴ and R⁵ are methyl.
